(19) United States
(12) Patent Application Publication
(10) Pub. No.: US 2026/0256531 A1
(43) Pub. Date: Sep. 3, 2026

(54) SYSTEMS AND METHODS FOR CONTROLLED AUTONOMOUS PHYSICAL INTERACTION IN MEDICAL PROCEDURES

(71) Applicant: Imagine Health Group Inc., Miami, FL (US)

(72) Inventors: Mohan Mahadevan, London (GB); Arshak Navruzyan, Paris (FR); Atabak Nezhadfard, London (GB); Vishnu Dev Amara, London (GB); Vitalii Pruks, London (GB)

(21) Appl. No.: 19/541,578

(22) Filed: Feb. 17, 2026

Related U.S. Application Data
(60) Provisional application No. 63/759,278, filed on Feb. 17, 2025.
(60) Provisional application No. 63/759,280, filed on Feb. 17, 2025.

(51) Int. Cl. A61B 34/32; A61B 34/00; A61B 34/20

(52) U.S. Cl. CPC A61B 34/32; A61B 34/20; A61B 34/25; A61B 2034/2055; A61B 2034/2063; A61B 2034/254

(57) ABSTRACT

A system and method are provided for conducting and governing autonomous and semi-autonomous physical interaction between a medical device and a human body during a medical procedure. The procedure is represented by a protocol defining procedural objectives, sequencing, and authorization conditions for autonomous operation. Physical interaction is executed in accordance with the protocol while authorization conditions are evaluated independently of device-level control. Autonomous operation is selectively enabled, limited, shared with a human operator, or revoked based on evaluated authorization conditions, including safety constraints, procedural context, data quality, system state, and human supervisory input. Acquired multimodal sensor data is processed to support execution and governance, including mapping into a unified coordinate system. One or more artificial intelligence models are used to analyze acquired data and generate candidate actions without directly authorizing execution. Independent safety monitoring is provided to restrict or interrupt operation when safety conditions are detected. The system supports staged, reversible autonomy and is applicable to diagnostic, interventional, and therapeutic procedures.

### RELATED APPLICATIONS

This application claims priority to U.S. Provisional Patent Application Nos. 63/759,278 and 63/759,280, both filed Feb. 17, 2025, the entirety of each of which is incorporated herein by reference.

The present application is related to U.S. Patent Application entitled “SYSTEMS AND METHODS FOR DYNAMIC CONTROL OF MEDICAL DEVICES BASED ON PATIENT-SPECIFIC OPERATIONAL PARAMETERS,” filed on even date herewith and claiming priority to the same provisional applications identified above. The disclosure of that application is incorporated herein by reference in its entirety for all purposes. The present application and the related application describe complementary but distinct aspects of controlled autonomous physical interaction in medical procedures, and each is intended to define independently patentable subject matter.

### TECHNICAL FIELD

This application relates generally to robotic medical systems and, more particularly, to systems and methods for robotic-and artificial-intelligence (AI)-assisted physical interactions (including but not limited to medical procedures) with human-in-the-loop (HITL) safety oversight. The disclosed techniques are applicable to a wide variety of medical procedures and clinical workflows, including diagnostic, interventional, invasive, and non-invasive procedures, and optionally involve imaging, sensing, therapeutic delivery, or procedural execution across diverse anatomical and procedural contexts.

### BACKGROUND

Medical procedures that involve physical interaction between a device and the human body are widely used across diagnostic, monitoring, therapeutic, and interventional medicine. In such procedures, a medical device must be physically positioned, manipulated, or applied to a patient in order to acquire diagnostic data or deliver a therapeutic effect. The manner in which this physical interaction is conducted directly impacts patient safety, data quality, procedural effectiveness, and clinical outcomes. These procedures are performed across a range of clinical environments including hospitals, outpatient clinics, emergency departments, mobile care units, pharmacies, and home-based settings.

A substantial subset of these procedures are non-invasive or minimally invasive and rely on controlled physical interaction rather than penetration of the body. Examples include diagnostic ultrasound imaging for cardiac, abdominal, obstetric, vascular, pulmonary, and musculoskeletal assessment; non-invasive physiological monitoring such as auscultation or automated placement of sensors for cardiac or pulmonary evaluation; image-guided localization and targeting procedures such as ultrasound-guided biopsy planning or injection guidance; non-invasive therapeutic procedures such as focused ultrasound therapy, shockwave therapy, vibration-based therapy, or external neuromodulation; emergency interventions such as cardiopulmonary resuscitation assistance; and rehabilitation or physical therapy procedures involving repeated guided physical interaction with a patient.

Many such procedures remain highly dependent on manual operation by trained clinicians. For example, diagnostic ultrasound imaging requires continuous adjustment of probe position, orientation, contact force, scan trajectory, and imaging parameters to obtain diagnostically useful views. These actions must be conducted in real time based on visual feedback and clinical judgment and depend heavily on operator experience and physical endurance. As a result, procedural quality and consistency vary significantly between operators, and repetitive or sustained physical tasks contribute to work-related musculoskeletal injury. Similar operator-dependent variability and physical burden are observed in other procedures that require precise or prolonged physical interaction with a patient. As a result, critical procedural decisions are often made through implicit, undocumented feedback loops that cannot be consistently reproduced, audited, or transferred across operators, clinical sites, or patient populations.

Recent advances in robotics, sensing technologies, and computer-based data processing have led to systems intended to assist clinicians in conducting such procedures. Existing systems include robotic arms configured to hold or position a medical device, software systems that provide guidance or quality feedback to an operator, or automated routines that conduct predefined motions or subtasks. While such systems reduce certain physical burdens or automate limited aspects of a procedure, they are typically confined to specific devices, sensing modalities, anatomical targets, or narrowly defined workflows. In many cases, autonomy is implemented as a fixed operating mode or as task-specific automation rather than as a process that is continuously governed during execution. In such systems, automation is typically implemented as a predefined operating mode or as a collection of task-specific routines, rather than as a continuously evaluated process that can be incrementally enabled, restricted, or revoked during execution of a procedure.

In regulated clinical environments, these limitations are particularly significant. Medical devices that physically interact with patients must operate within strict safety, risk management, and accountability constraints. Autonomous or semi-autonomous physical interaction must therefore be both properly conducted and explicitly governed. It is not sufficient for a system to be capable of executing motions or interactions; it must also determine when such actions are authorized, under what conditions they are allowed to proceed, how safety constraints are enforced, and how control is shared with or transferred to a human operator during a procedure. In regulated clinical environments, autonomy must therefore be explicitly authorized, bounded, and auditable throughout execution of a procedure. Systems that embed autonomy decisions directly within low-level control logic make it difficult to demonstrate, verify, or adjust such authorization in a manner consistent with regulatory, safety, and accountability requirements.

Existing systems generally do not provide a unified framework that separates the mechanisms used to conduct physical interaction from the higher-level logic used to govern autonomy. Instead, prior approaches often conflate low-level motion or control implementation with autonomy decision-making. As a result, authorization of autonomous physical interaction is tightly coupled to specific robotic platforms, control algorithms, or sensor configurations, limiting general applicability across different procedures, devices, and clinical environments and making it difficult to adapt autonomy behavior dynamically during a procedure. This coupling of autonomy decision-making with low-level control implementation prevents such systems from dynamically adapting autonomy behavior in response to changing procedural context, human input, or safety conditions, and inhibits reuse of autonomy logic across different devices, procedures, or clinical environments.

Moreover, existing systems generally lack a mechanism for representing procedural state in a manner that is independent of device control implementation. As a result, autonomy decisions are not explicitly tied to procedural phase, clinical intent, or authorization context, but instead are inferred indirectly from control mode or operator input.

Accordingly, there exists a need for a technical solution that decouples the execution of physical interaction from the governance of autonomy, enabling authorization, limitation, sharing, and revocation of autonomous behavior to be evaluated and enforced independently of device-specific control mechanisms. Such a solution must not only enable a device to execute physical interaction according to a defined procedure but must also explicitly determine when such interaction is authorized, limited, shared with a human operator, or revoked. In clinical practice, this determination depends on dynamic inputs including human supervisory approval, procedural protocol requirements, patient-specific conditions, and system state or safety feedback.

Further, autonomy in such systems must be governed as a staged and reversible process rather than as a fixed operating mode. Authorization to perform autonomous physical interaction can be granted incrementally, restricted to specific procedural steps or regions, shared between automated and human control, or revoked entirely based on real-time evaluation of authorization conditions. These conditions can change during execution of a procedure and incorporate human feedback, data quality assessment, protocol compliance, or safety-related events. Existing systems do not provide a unified, device-agnostic framework for conducting physical interaction while simultaneously governing autonomy in this manner across diverse non-invasive and minimally invasive diagnostic and therapeutic procedures.

### Challenges in Ultrasound Imaging as an Illustrative Clinical Context

Ultrasound imaging is widely used in clinical practice due to its non-invasive nature, absence of ionizing radiation, real-time imaging capability, and broad applicability across medical specialties. As a result, ultrasound is frequently employed for diagnostic evaluation, procedural guidance, and longitudinal monitoring in diverse clinical environments. However, despite its widespread adoption, acquisition of high-quality ultrasound data remains highly dependent on operator skill and physical interaction between a human operator and a patient.

The process of acquiring diagnostically useful ultrasound images is both cognitively complex and physically demanding. Image formation depends on acoustic interactions that vary with patient anatomy, tissue composition, probe orientation, contact force, and device configuration, requiring continuous adjustment and interpretation by the operator. These factors must be optimized jointly and in real time, often over extended periods, which places significant physical and cognitive demands on clinicians performing ultrasound examinations.

These characteristics give rise to several persistent challenges. First, sustained manual manipulation of ultrasound probes contributes to a high incidence of work-related injury among ultrasound practitioners. Second, ultrasound image quality and diagnostic reliability vary significantly across operators, leading to inconsistent clinical outcomes and, in some cases, the need for repeat or alternative imaging procedures. Third, acquisition of ultrasound data requires specialized training and expertise, resulting in workforce shortages and limited scalability across institutions and geographic regions. Fourth, ultrasound procedures typically require the physical presence of a qualified operator, constraining access to care in remote, underserved, or resource-limited settings.

In response to these challenges, a variety of approaches have been explored to augment or automate ultrasound data acquisition. These approaches generally fall into several categories, including teleguided ultrasound with remote human supervision, AI-based guidance systems that assist a local operator, teleoperated robotic ultrasound systems, and fully autonomous robotic ultrasound research platforms. While each of these approaches addresses certain aspects of the problem space, none provides a comprehensive, scalable, and clinically robust solution.

For example, teleguided ultrasound systems rely on real-time guidance from a remote expert and therefore do not reduce dependence on specialized personnel or address inter-operator variability. AI-based guidance systems assist with limited subsets of anatomy or imaging tasks but typically lack mechanisms to manage uncertainty, adapt to pathological variation, or govern long-tail failure cases. Teleoperated robotic ultrasound systems remove the need for physical proximity but introduce additional complexity, cost, latency, and scalability constraints, while remaining dependent on remote expert control. Research efforts toward fully autonomous robotic ultrasound have demonstrated feasibility in controlled settings, but have not translated into clinically viable systems capable of safe, adaptive operation across diverse patient populations and procedural contexts.

A common limitation of these prior approaches is the absence of a unified framework that separates execution of physical interaction from governance of autonomy. In particular, existing systems lack mechanisms for staged, reversible authorization of autonomous operation that integrates continuous safety monitoring, procedural context, and human supervisory input. As a result, autonomy is either tightly constrained, narrowly scoped, or insufficiently governed to support safe and scalable deployment in real-world clinical environments.

### SUMMARY

The present invention provides a generalized, device-agnostic technical framework for conducting and governing autonomous and semi-autonomous physical interaction between a diagnostic sensor or interventional medical device and a human body during a medical procedure. Unlike prior approaches that treat autonomy as a fixed operating mode or embed autonomy decisions within device-specific control logic, the disclosed methods and systems treat autonomy as a protocol-driven, conditionally-authorized, and dynamically governed process. Physical interaction with a patient is executed only under explicitly evaluated authorization conditions and is continuously supervised throughout procedural execution. Autonomous physical interaction is permitted only when such authorization conditions are satisfied and is otherwise restricted, modified, shared with a human operator, escalated, degraded, or revoked.

In accordance with the invention, a medical procedure is conducted according to a protocol that specifies intended procedural objectives, data acquisition or intervention targets, sequencing requirements, and permissible conditions under which autonomous physical interaction can occur. The protocol corresponds to a standardized clinical procedure or to a clinician-defined procedure tailored to a specific patient, indication, or clinical objective. The protocol serves as a shared, modality-independent representation that informs both execution of physical interaction and governance of autonomy, and the procedure can be predefined prior to execution or dynamically updated during execution.

The method further comprises conducting the protocol through controlled physical interaction between one or more medical devices (including, but not limited to, diagnostic sensors, therapeutic devices, interventional devices) and the human body. Such physical interaction includes positioning, navigating, orienting, actuating, or otherwise applying a device relative to a patient to acquire diagnostic data, perform an intervention, or deliver a therapeutic effect. The manner and extent of autonomous physical interaction can be incrementally authorized, bounded to specific procedural steps or regions, or shared with a human operator based on evaluated authorization conditions.

The method further comprises acquiring data from a plurality of sensing elements configured to observe one or more of the medical device, the patient, the surrounding environment, and procedural outcomes during execution of the protocol. The sensing elements can be integrated with the device, disposed on the device, on an end effector, positioned externally to the device, or located in the clinical environment. The disclosed framework is independent of sensor modality, placement, manufacturer, or configuration, enabling applicability across diverse devices and procedures.

Based on the acquired data, one or more computational processes are executed to support both execution of the procedure and governance of autonomy. Such processes can generate candidate actions, procedural state assessments, quality indicators, or predicted outcomes associated with execution of the protocol. One or more such outputs are presented for evaluation or authorization prior to initiating or continuing autonomous physical interaction. The computational processes include rule-based logic, model-based control, optimization routines, data-driven models, machine learning techniques, or combinations thereof.

The method further comprises evaluating authorization conditions using a decision engine that is logically separated from low-level device control. Authorization conditions include safety constraints, protocol-defined requirements, procedural state, data quality criteria, patient-specific parameters, system health or fault indicators, and human supervisory input. Authorization conditions can be evaluated prior to initiating autonomous physical interaction, at discrete procedural checkpoints, and continuously during execution of the protocol.

When authorization conditions are satisfied, autonomous physical interaction is conducted in accordance with the protocol under a determined level of autonomy. When authorization conditions are not satisfied, the method governs autonomy by restricting physical interaction, modifying autonomous behavior, transitioning to shared control, escalating to increased human involvement, or revoking autonomous operation entirely. Authorization is re-evaluated dynamically, enabling staged, reversible, and context-dependent control of autonomy throughout the procedure.

Human supervisory control is integrated as a core component of the governance framework. A human operator can authorize, limit, modify, or revoke autonomous physical interaction at any time during execution of the protocol. Human supervisory input is provided locally or remotely and can be treated as an explicit authorization condition by the decision engine, enabling transparent, auditable, and clinically accountable control of autonomous physical interaction.

It should be appreciated that, in some embodiments, the disclosed governance framework extends beyond advisory or decision-support functionality and operates to actively mediate physical control authority within the system. In particular, human-initiated control inputs—including teleoperated commands, shared-control adjustments, or manual positioning directives—are not transmitted directly to the robotic device 206 or procedural device 208 without evaluation. Instead, such inputs are processed by the autonomy control layer and evaluated against the procedural protocol, current authorization conditions, and safety constraints prior to actuation. Human-generated commands are therefore conditioned (e.g., generated, constrained, filtered, modified, and/or rejected) by the system when inconsistent with protocol-defined requirements, patient-specific parameters, or monitored safety boundaries. As a result, physical device state transitions occur only when authorized within the governance framework, regardless of whether the initiating command originates from an automated process or a human operator. This architecture preserves separation between low-level device control and autonomy governance while ensuring that all electromechanical transformations remain bounded by explicitly evaluated authorization and safety conditions.

In some embodiments, the disclosed system is architecturally configured such that no physical actuation of a robotic device or procedural device can occur unless authorization conditions have been evaluated and satisfied by a governance engine that is logically distinct from low-level device control logic. The governance engine operates as a mandatory mediation layer between any source of candidate control input—including autonomous algorithms, shared-control routines, teleoperation commands, or direct human input—and actuator-level control signals. Human-generated commands are not transmitted directly to robot actuators or procedural device actuators; instead, they are conditioned, constrained, filtered, modified, or rejected based on protocol-defined limits, patient-specific operational bounds, safety thresholds, and currently valid authorization states before propagation to the device control module.

In some embodiments, actuator drivers are configured to execute control signals only when accompanied by a valid authorization output generated by the governance engine, thereby structurally gating electromechanical state transitions at the signal level. Accordingly, the disclosed system is not limited to advisory or decision-support functionality. Even in manual or shared-control modes, human inputs remain bounded within a defined operational envelope and are subject to dynamic constraint enforcement, including limitation of degrees of freedom, force ceilings, workspace boundaries, and protocol-defined regions of operation. The framework therefore directly governs tangible electromechanical transformation at the device-patient interface and does not merely generate or display recommendations.

In accordance with another aspect of the invention, a system is provided for conducting and governing autonomous and semi-autonomous physical interaction between a medical device and a human body during a medical procedure. The system comprises one or more medical devices configured to physically interact with a patient; a plurality of sensing elements configured to acquire data relating to the medical device, the patient, procedural context, or the surrounding environment; one or more computing elements configured to represent and execute a procedure protocol, generate candidate actions, evaluate authorization conditions, and govern levels of autonomy during execution; and one or more interfaces configured to receive human supervisory input. The disclosed system architecture is independent of any particular robotic platform, device form factor, probe configuration, sensor modality, control algorithm, anatomical target, or clinical environment, thereby enabling reuse across diverse procedures and care settings.

In accordance with a further aspect of the invention, a non-transitory computer-readable medium is provided comprising instructions that, when executed by one or more processors, cause the system to conduct and govern autonomous and semi-autonomous physical interaction in accordance with the methods described herein. The instructions implement protocol representation, sensing integration, candidate action generation, authorization evaluation, autonomy governance, and human supervisory interaction, without limitation as to specific software architecture or execution environment.

In one non-limiting exemplary embodiment, the method and system are applied to an ultrasound imaging procedure. In this embodiment, the procedure protocol corresponds to a standardized or clinician-defined ultrasound examination, and the medical device comprises an ultrasound probe configured to be physically positioned and manipulated relative to a patient. The system conducts ultrasound scanning while dynamically governing transitions among human-guided, shared-control, semi-autonomous, and autonomous operation based on evaluated authorization conditions. Safety-bounded physical interaction is maintained throughout execution, with immediate human supervisory control available at all times. This ultrasound embodiment is provided as an illustrative instantiation of the disclosed framework and does not limit the scope of the invention.

By unifying protocol-driven execution of physical interaction with explicit, staged, and reversible governance of autonomy, the invention provides a scalable, device-agnostic, and regulator-compatible technical framework for conducting and supervising autonomous and semi-autonomous medical procedures. The disclosed framework enables consistent authorization, supervision, and accountability across a wide range of non-invasive and minimally invasive diagnostic, interventional, and therapeutic applications. Also, by decoupling autonomy governance from device-level control logic, the disclosed architecture reduces cross-dependencies between motion control and authorization logic, enables reuse of governance logic across heterogeneous devices, supports independent verification of authorization decisions, and improves safety assurance by preserving independent watchdog enforcement layers.

The invention, in another aspect, features a system for performing a medical procedure involving physical interaction with a patient. The system includes a robotic device having a plurality of actuated joints and configured to position a procedural instrument relative to a patient. The system also includes one or more sensors configured to acquire, during the medical procedure, sensor data comprising at least one of device pose data, interaction force data, or imaging data associated with physical interaction between the procedural instrument and the patient. The system also includes one or more processors operatively coupled to the robotic device and the one or more sensors. The one or more processors are configured to execute a procedural protocol defining an ordered set of procedural steps and permitted ranges of physical interaction between the procedural instrument and the patient. The one or more processors are configured to evaluate authorization conditions based on the acquired sensor data and the procedural protocol. The one or more processors are configured to control actuation of the robotic device based on the evaluated authorization conditions to selectively (a) permit automated movement of the procedural instrument, (b) limit automated movement of the procedural instrument to a subset of degrees of freedom, (c) transition control of the procedural instrument to shared control with a human operator, or (d) suspend automated movement of the procedural instrument. The system also includes a user interface configured to receive supervisory input from a human operator for use in evaluating the authorization conditions or controlling actuation of the robotic device.

The invention, in another aspect, features a method of conducting a medical procedure involving physical interaction between a medical device and a patient. A robotic device is controlled to position and move a procedural instrument relative to a patient. Sensor data is acquired during the medical procedure, the sensor data comprising at least one of device pose data, interaction force data, or imaging data associated with physical interaction between the procedural instrument and the patient. A procedural protocol is executed, the procedure protocol defining an ordered set of procedural steps and permitted ranges of physical interaction between the procedural instrument and the patient. One or more processors evaluate authorization conditions based on the acquired sensor data and the procedural protocol. Actuation of the robotic device is controlled based on the evaluated authorization conditions to selectively (a) permit automated movement of the procedural instrument, (b) limit automated movement of the procedural instrument to a subset of degrees of freedom, (c) transition control of the procedural instrument to shared control with a human operator, or (d) suspend automated movement of the procedural instrument.

Any of the above aspects can include one or more of the following features. In some embodiments, the authorization conditions include one or more of interaction force thresholds, positional constraints, imaging quality criteria, protocol-defined completion states, or combinations thereof. In some embodiments, the one or more processors are configured to evaluate the authorization conditions continuously during movement of the procedural instrument. In some embodiments, the robotic device is configured to position the procedural instrument according to a six-degree-of-freedom pose comprising translation along x-, y-, and z-axes and rotation about roll, pitch, and yaw axes. In some embodiments, the one or more processors are configured to modify the procedural protocol during the medical procedure in response to sensor data or supervisory input from the human operator.

In some embodiments, the one or more processors are configured to generate candidate control actions using a trained machine learning model based on the acquired sensor data. In some embodiments, the one or more processors are configured to subject the candidate control actions to evaluation of the authorization conditions prior to controlling actuation of the robotic device.

In some embodiments, a safety monitoring module independently monitors interaction forces and suspends automated movement of the procedural instrument when a safety threshold is exceeded. In some embodiments, the safety monitoring module interrupts automated movement independently of execution of the procedural protocol.

In some embodiments, the one or more processors are configured to map imaging data and device pose data into a unified coordinate system used to guide subsequent positioning of the procedural instrument. In some embodiments, the procedural instrument comprises an ultrasound probe and the medical procedure comprises a diagnostic ultrasound imaging procedure.

### BRIEF DESCRIPTION OF THE DRAWINGS

The advantages of the invention described above, together with further advantages, may be better understood by referring to the following description taken in conjunction with the accompanying drawings. The drawings are not necessarily to scale, emphasis instead generally being placed upon illustrating the principles of the invention.

FIG. 1 is a schematic diagram illustrating an example protocol-driven framework for defining and executing an autonomous or semi-autonomous medical procedure in accordance with the present invention.

FIG. 2 is a schematic diagram illustrating an example system configuration for conducting and governing autonomous physical interaction with a patient during a medical procedure.

FIG. 3 is a detailed block diagram of a computing device in the system of FIG. 2.

FIG. 4 is a flow diagram of an exemplary method for generating a training dataset to be used in training one or more artificial intelligence (AI) models.

FIG. 5 is a flow diagram illustrating an exemplary method for training an AI model using a training dataset.

FIG. 6 is a detailed block diagram of the safety monitoring module of FIG. 3.

FIG. 7 is a flow diagram of an exemplary method of autonomy and governance with human-in-the-loop and safety monitoring checkpoints.

FIG. 8 is an exemplary process for generating a reference database for a selected organ system in the context of ultrasound imaging.

FIG. 9 is an exemplary workflow process for an initial scan-time acquisition.

FIG. 10 is an exemplary workflow process for scan-time procedural flow.

FIG. 11 is a diagram of an exemplary workflow process of planning and updating a scan path based upon a joint embedding manifold.

FIG. 12 is a diagram illustrating deterministic geometric estimation of an acoustic window using two-dimensional (2D) and three-dimensional (3D) imaging data.

FIG. 13 is a diagram illustrating determination of an optimal robot/probe location and orientation for an anchor view.

FIG. 14 is a diagram illustrating a reference optimal probe pose and controlled perturbations of the probe pose.

### DETAILED DESCRIPTION

In the following description, embodiments of the disclosed system and methods are described with reference to the accompanying drawings, which illustrate representative implementations of the invention. Specific terminology is used for clarity and descriptive purposes; however, the disclosure is not intended to be limited to the particular terms or examples described. Rather, each described component, feature, or operation is intended to encompass technical equivalents that perform similar functions or achieve similar results.

The following description presents exemplary embodiments of a generalized, device-agnostic method and system for conducting and governing autonomous and semi-autonomous physical interaction between a medical device and a human body during a medical procedure. The embodiments described herein are illustrative and non-limiting, and the invention is not restricted to the specific configurations, components, control mechanisms, or procedural sequences described.

As used herein, autonomy refers to execution of physical interaction by a device without continuous direct manual control by a human operator, which can occur in whole or in part and under supervisory oversight. Governance refers to a logically distinct process for determining whether, when, and under what conditions such autonomous physical interaction is authorized, constrained, shared with a human operator, escalated, degraded, or revoked during execution of a procedure.

### Overall Framework

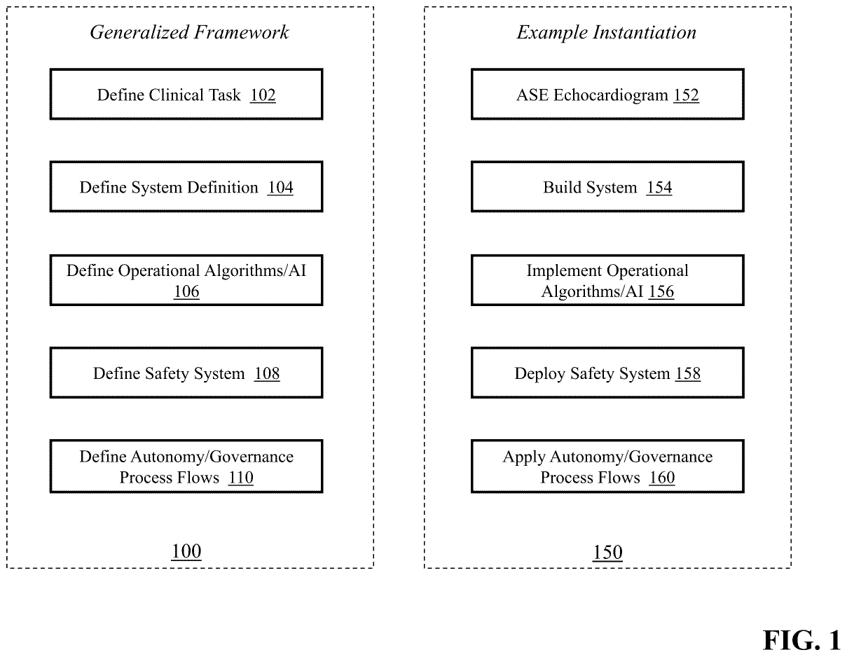

FIG. 1 is a schematic diagram illustrating an example protocol-driven framework for defining and executing an autonomous or semi-autonomous medical procedure in accordance with the present invention. The left-hand portion of FIG. 1 illustrates a generalized, procedure-independent framework 100 applicable across medical modalities and clinical contexts, while the right-hand portion illustrates a non-limiting instantiation 150 of the generalized framework 100 for a specific standardized clinical protocol (i.e., an echocardiogram ultrasound procedure performed according to the American Society of Echocardiography (ASE) protocol). The illustrated instantiation is provided solely for explanatory purposes and does not limit the scope of the disclosed framework to any particular protocol, modality, anatomical target, or clinical environment. The illustrated right-hand instantiation 150 demonstrates how a specific clinical protocol is mapped onto the generalized framework 100 of the invention, while preserving separation between procedural definition, system execution, safety monitoring, and autonomy governance.

As shown in FIG. 1, the generalized framework 100 includes functions for defining a procedural task or protocol (102), defining a system configuration for performing the procedure (104)—including hardware, robotics, algorithms, and human-in-the-loop (HITL) elements, provision of algorithmic, agentic, and control computational pipelines (106), specifying safety and monitoring functions (108), and defining HITL process flows for authorization, supervision, and control of autonomous operation (110). It should be appreciated that the provisioning of HITL process flows and elements are optional in some embodiments.

Also, it should be appreciated that the generalized framework 100 of FIG. 1 represents a logical decomposition of system functions rather than a required temporal sequence. In some embodiments, one or more of the illustrated functions 102, 104, 106, 108, 110 can be defined, instantiated, modified, or evaluated prior to, during, or after execution of a medical procedure, and certain elements can be iteratively refined or dynamically updated based on procedural context, system state, or human supervisory input.

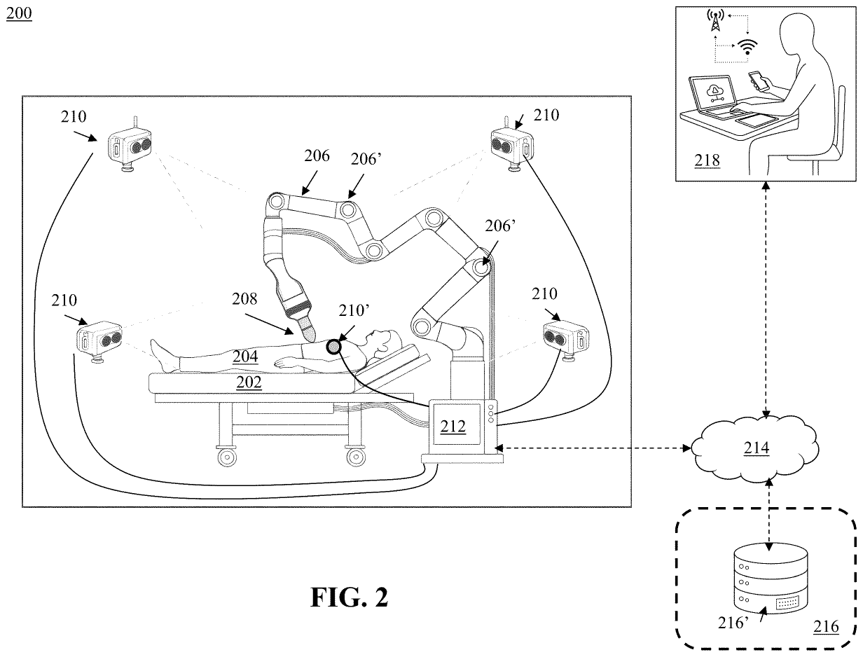

As mentioned above, the non-limiting instantiation 150 of the generalized framework 100 corresponds to a procedural task (e.g., echocardiogram ultrasound) based upon a predefined clinical protocol (ASE) (element 152). At element 154 of the instantiation 150, the clinical protocol is associated with a corresponding system definition that specifies, for the procedure, an appropriate combination of medical devices, sensing elements, robotic or actuated components, computational resources, and HITL interfaces. For example, FIG. 2 (described below) illustrates one example of a complete system configuration 200 suitable for executing an echocardiogram ultrasound under the ASE protocol, although alternative system definitions can be used without departing from the disclosed framework.

At element 156, the instantiation 150 further defines an association of the ASE protocol with an algorithmic and data processing architecture implemented in a computing device configured to support execution of the procedure. This architecture includes acquisition and processing of sensor data, execution of AI or agentic control algorithms, and generation of procedural outputs, candidate actions, or quality indicators relevant to the protocol.

At element 158, the instantiation 150 further implements one or more safety and monitoring functions that applicable to the protocol. The safety and monitoring functions can monitor system state, sensed data, and procedural constraints to determine whether autonomous physical interaction remains within permitted conditions defined by the protocol and applicable authorization criteria.

At element 160, the instantiation 150 incorporates human expert involvement in governing autonomy during execution of the protocol. For example, HITL processes are used to evaluate authorization conditions, approve or limit autonomous behavior, escalate to increased human involvement, or revoke autonomy entirely. These processes operate in coordination with automated decision logic and are not limited to a specific clinical role, physical location, or mode of interaction.

Also, the framework 100 of FIG. 1 logically separates execution of physical interaction from governance of autonomy. Execution encompasses physical interaction between a medical device and a patient, which is performed under human guidance, shared control, or autonomous control, while governance determines whether, when, and under what conditions such execution is authorized, constrained, shared, or revoked during performance of the procedure.

### System Configuration

FIG. 2 is a schematic diagram illustrating an example system configuration 200 for conducting and governing autonomous physical interaction with a human body during a medical procedure. The system configuration 200 includes a patient support structure 202 (e.g., a bed, table, or other apparatus) for supporting a patient 204 during the medical procedure, a robotic device 206, one or more procedural devices 208 (which are typically affixed to the robotic device 206) configured to perform functions pertaining to execution of the medical procedure on the patient, one or more environment sensors 210 arranged to view and capture data pertaining to the patient, the robot, and/or the surrounding clinical environment, a computing device 212 coupled to the robotic device 206 (and optionally the patient support structure 202), the procedural devices 208, the environment sensors 210, and a communications network 214 connecting the computing device 212 to one or more external computing resources, such as a cloud computing environment 216 including one or more data stores (e.g., database 216′) and a remote computing device 218 operated by a human expert.

The robotic device 206 can comprise any actuated mechanical system configured to position, orient, move, or otherwise manipulate one or more procedural devices 208 (e.g., medical devices, tools, or end effectors) relative to the patient support structure 202 and/or the patient 204. In some embodiments, the robotic device 206 includes one or more articulated joints, links, linear or rotary actuators, or compliant elements providing one or more degrees of freedom, and can be configured to execute commanded motions, trajectories, forces, or interaction states under control of computing device 212. For example, each joint of the robotic device 206 can include, be coupled to, or be associated with one or more sensing elements configured to measure or estimate joint state and interaction parameters. Such sensing elements include, but are not limited to, position or displacement sensors, rotary or linear encoders, velocity or speed sensors, accelerometers, gyroscopes, inertial measurement units, force or torque sensors, strain or load sensors, compliance or deflection sensors, temperature sensors, or combinations thereof. In some embodiments, joint parameters are measured directly, while in other embodiments one or more parameters are inferred or estimated using actuator signals, dynamic models, or state observers. It should be appreciated that any type, number, placement, and/or modality of joint-level sensing elements can be incorporated into the system 200 without limiting the scope of the technology described herein.

The robotic device 206 can be controlled at one or more abstraction levels, including joint-level control, task-space control, impedance or admittance control, force or torque control, or higher-level interaction objectives. It should be appreciated that control commands received at the robotic device 206 from the computing device 212 can specify desired positions, velocities, forces, compliance parameters, or interaction constraints—rather than explicit joint motions. The robotic device 206 is not limited to a particular form factor and comprises, by way of example and without limitation: an articulated robotic arm, a serial or parallel manipulator, a gantry-based system, a mobile robotic platform, a wearable or body-mounted actuator, or a combination thereof. The robotic device 206 can be fixed, movable, or repositionable relative to the patient support structure 202, the patient 204, and/or the clinical environment.

As mentioned above, the robotic device 206 can also include one or more procedural devices 208 that are configured to physically interact with the patient 204. In the example robotic device 206 of FIG. 2, the procedural devices 208 are positioned at one end of the robotic device 206 closest to the patient 204. Procedural devices 208 can comprise any diagnostic, interventional, or therapeutic apparatus configured to be mechanically coupled to, supported by, or manipulated by the robotic device 206 for performing physical interaction with the patient 204. In some embodiments, the procedural device 208 can be affixed to an end effector of a robotic arm or otherwise operatively coupled to the robotic device, and the device 208 is configured to acquire diagnostic data, perform an intervention, deliver a therapeutic effect, or combinations thereof. Also, it should be appreciated that the procedural device 208 can be rigidly mounted, removably attached, or dynamically coupled to the robotic device 206, and include mechanical, electrical, optical, fluidic, or wireless interfaces. In some embodiments, the procedural device 208 is interchangeable, allowing different procedural devices to be mounted to the same robotic platform without modification of the robotic device 206 itself.

Diagnostic procedural devices can include, but are not limited to, imaging probes, physiological sensing devices, localization or mapping instruments, or other devices configured to acquire diagnostic data through physical interaction with a patient. Interventional procedural devices include devices configured to penetrate, traverse, manipulate, or otherwise interact with tissue, including needles, catheters, cannulas, guidewires, biopsy tools, or similar instruments. Therapeutic procedural devices include devices configured to deliver energy, substances, or mechanical effects to tissue for treatment or modulation of a physiological condition. In some embodiments, a procedural device 208 performs multiple functions, such as acquiring diagnostic data while performing an intervention or delivering therapy, and transitioning between diagnostic, interventional, and therapeutic operation during execution of a medical procedure. Also, the procedural device 208 can be configured to engage in physical interaction with a patient involving contact force, pressure, penetration depth, orientation, motion trajectory, or applied energy, and such interaction parameters are monitored, controlled, or constrained by the controls and/or governance implemented by the computing device 212 and/or the remote device 218 of the human expert.

As described above, the system 200 also includes one or more environment sensors 210. As used herein, environment sensors 210 can comprise one or more sensing elements positioned in, on, or around a clinical environment and configured to acquire data relating to the patient support structure 202, the patient 204, the robotic device 206, the one or more procedural devices 208, human operators (e.g., technicians or other staff that may be present in the clinical environment), and/or environmental conditions (e.g., sound, temperature) during execution of a medical procedure. It should be appreciated that environment sensors 210 can be fixed, movable, or reconfigurable, and can be positioned on walls, ceilings, floors, carts, stands, booms, furniture, wearable mounts, or other structures within the clinical environment. In some embodiments, one or more environment sensors 210 can be integrated into existing clinical infrastructure.

Environment sensors 210 can comprise any of a number of different types, function sets, form factors, or modalities—such as visual sensors, audio sensors, environmental condition sensors, spatial/motion sensors, or presence/identification sensors—depending upon the requirements of the system 200, the medical procedure and/or the clinical environment. Visual environment sensors can include cameras or imaging devices configured to capture images or video of the patient support structure 202, the patient 204, the robotic device 206, the procedural devices 208, or other aspects of the clinical workspace. Audio environment sensors can include microphones or acoustic sensors configured to acquire sound data relating to speech, alarms, mechanical operation, or patient vocalizations. Environmental condition sensors can be configured to measure ambient conditions within the clinical environment, including temperature, humidity, lighting, or air quality. Spatial environment sensors can include range, proximity, or motion sensors configured to detect relative positions or movement of objects, people, or devices within the clinical environment. Presence or identification sensors can be used to detect or identify patients, clinicians, or other personnel within the clinical environment.

In some embodiments, one or more of the environment sensors 210 comprise physiological sensing elements such as electrocardiogram (ECG) leads (210′) configured to acquire electrical activity of the patient's heart during execution of the medical procedure. The ECG leads 210′ may be positioned on the patient's body and operatively coupled to the computing device 212 directly (or via a monitoring system). Signals acquired from the ECG leads 210′ can be sampled, digitized, and transmitted to the computing device 212 for processing in real time. The computing device 212 may use information derived from the ECG signal data—such as heart rate, cardiac cycle phase, rhythm irregularities, or detected trigger events—to inform or adjust the context of the medical procedure, modify operational parameters of the procedural device 208, or synchronize data acquisition to specific phases of the cardiac rhythm.

In some embodiments, data acquired from environment sensors 210 can be used to determine procedural context, monitor safety conditions, detect unexpected events, support authorization decisions, or modify or constrain autonomous physical interaction in real time. Also, in some embodiments, data from environment sensors 210 can be aggregated, fused, or integrated with data from patient support structure-mounted sensors, robotic device-mounted sensors (e.g., procedural sensors 208), joint-level sensors (e.g., 206′), and/or patient-mounted sensors to generate a unified representation of procedural state. In some embodiments, environmental parameters can be estimated or inferred from environmental sensor data using computational models rather than measured directly.

Environment sensors 210 are operatively coupled to computing device 212 for communication of acquired data. Such coupling can be implemented using wired or wireless communication links, including direct connections, networked connections, or combinations thereof. In some embodiments, environment sensors 210 transmit raw sensor data, processed sensor data, metadata, or event signals to the computing device 212 for use in execution of the medical procedure and governance of autonomous physical interaction. Communication can occur continuously, periodically, on demand, or in response to detected events, and can utilize any suitable communication protocol, interface, or transport mechanism.

Computing device 212 includes specialized hardware and/or software modules that execute on one or more processors and interact with memory modules of computing device 212, to receive data from other components of system 200, transmit data to other components of system 200, and perform functions including but not limited to, data acquisition, data processing, data transmission, command generation, and other tasks relating to execution of a medical procedure. The computing device 212 receives data indicative of physical interaction between one or more devices (e.g., procedural devices 208) and a patient, procedural context, environmental conditions (e.g., from environment sensors 210), system state, or patient state, and the computing device 212 can use such data to support execution, monitoring, and governance of autonomous and semi-autonomous physical interaction. As mentioned above, the computing device 212 can be communicatively and/or operatively coupled to the patient support structure 202, the robotic device 206, the procedural devices 208, and the environment sensors 210 for the purpose of acquiring, processing, transmitting, and managing data associated with execution of a medical procedure. Generally, computing device 212 is configured to execute one or more software modules to perform its designated functions. In some embodiments, the computing device 212 can be implemented as a single computing unit (e.g., located in the clinical environment) or as a distributed computing architecture comprising multiple computing nodes, controllers, or processing modules. Although FIG. 2 depicts a single computing device 212 in the clinical environment, it should be appreciated that any number of computing devices, arranged in a variety of architectures, resources, and configurations (e.g., cluster computing, virtual computing, cloud computing) can be used without departing from the scope of the technology described herein.

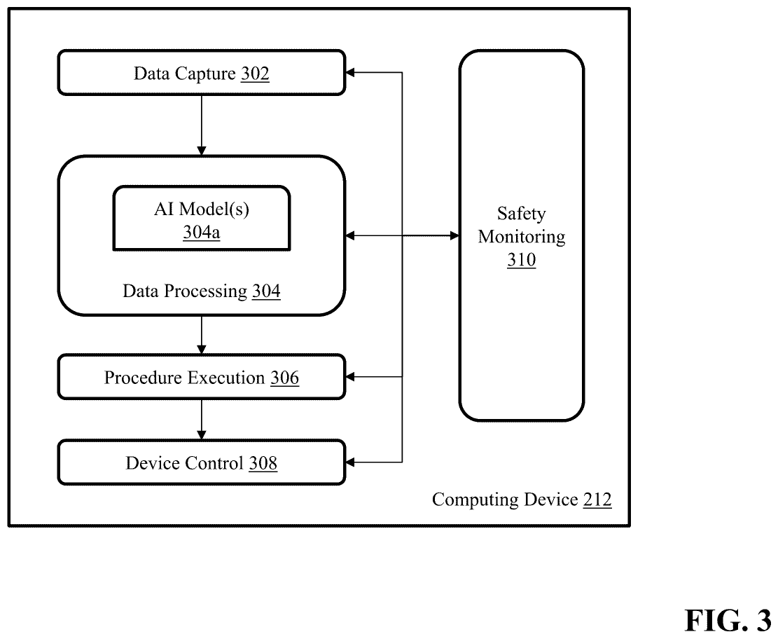

FIG. 3 is a detailed block diagram of the computing device 212 of FIG. 2. As shown in FIG. 3, the computing device 212 includes a data capture module 302, a data processing module 304 with one or more AI models 304a, a procedure execution module 306, a device control module 308, and a safety monitoring module 310. In some embodiments, modules 302, 304, 306, 308, and 310 are specialized sets of computer software instructions programmed onto one or more dedicated processors in computing device 212 and can include specifically designated memory locations and/or registers for executing the specialized computer software instructions.

Although modules 302, 304, 306, 308, and 310 are shown in FIG. 3 as executing within a single computing device 212, in some embodiments the functionality of modules 302, 304, 306, 308, and 310 can be distributed among a plurality of computing devices (including, but not limited to, one or more computing resources in cloud computing environment 216). Computing device 212 enables modules 302, 304, 306, 308, and 310 to communicate with each other in order to exchange data for the purpose of performing the described functions.

In some embodiments, data capture module 302 is configured to acquire data from other devices/components of system 200 relating to execution of a medical procedure. The data captured by module 302 includes, but is not limited to, data from environment sensors 210, procedural devices 208, robotic device sensors 206a′, patient support structure 204, and/or other user input devices. Such data includes images, video, audio signals, force or torque measurements, device pose or motion data, physiological signals, environmental measurements, timestamps, and metadata. The data capture module 302 can operate continuously, periodically, or be event-driven, and the module 302 can perform functions such as time synchronization, buffering, preprocessing, or formatting of acquired data for downstream processing.

In some embodiments, data processing module 304 is configured to process data acquired by the data capture module 302 to support execution, monitoring, and adaptation of the medical procedure. Data processing module 304 can perform one or more computational operations including filtering, normalization, feature extraction, sensor fusion, state estimation, and transformation of raw sensor data into representations suitable for analysis or decision support. The data processing module 304 can operate in real time or near real time, and module 304 generates intermediate outputs used by AI models (e.g., AI model(s) 304a), procedure execution logic, or safety monitoring components.

Data processing module 304 includes one or more AI models 304a, each of which comprises a trained model/algorithm/agent configured to analyze processed data provided by the data processing module 304. The AI models 304a are configured to perform a number of different data analysis tasks including, without limitation, perception, procedural state estimation, prediction, quality assessment, anomaly detection, or generation of candidate actions or recommendations. The AI model 304a can receive inputs such as sensor-derived features, device state information, protocol parameters, or contextual data that originate at one or more other components of system 200, and the model 304a can produce outputs including inferred procedural states, estimated parameters, confidence measures, quality scores, or suggested next procedural steps. The AI model 304a generates outputs that can be provided to procedure execution module 306 and/or to safety monitoring module 310 for further processing. It should be appreciated that in a typical embodiment, the output from AI model 304a is not used by itself to authorize physical interaction with a patient.

Procedure execution module 306 is configured to manage execution of the medical procedure in accordance with the defined clinical protocol and operational process workflow. The procedure execution module 306 can use outputs generated by the data processing module 304/AI model(s) 304a to determine how the procedure should progress, including sequencing of procedural steps, adjustment of operational state, or selection of candidate actions. In some embodiments, the procedure execution module 306 operates under different levels of autonomy, including human-guided, shared-control, semi-autonomous, or autonomous execution, subject to authorization and safety constraints imposed by other system components.

Device control module 308 is configured to generate and transmit control commands to one or more devices of system 200 that are either involved in, monitoring, or acquiring data relating to the medical procedure, including patient support structure 202, patient-wearable devices, robotic device 206 and corresponding sensing elements 206', procedural devices 208, and environment sensors 210. In some embodiments, device control module 308 translates high-level execution directives or candidate actions received from procedure execution module 306 into low-level control signals such as motion commands, force or torque limits, compliance parameters, or actuator setpoints. Also, device control module 308 can operate in conjunction with real-time feedback from patient support structure 202, patient-wearable devices, robotic device 206 and corresponding sensing elements 206′, procedural devices 208, and environment sensors 210, and device control module 308 can be configured to enforce control constraints defined by the procedure execution module 306 or safety monitoring module 310.

Safety monitoring module 310 is configured to independently monitor system operation and physical interaction conditions to detect safety-relevant events during execution of the medical procedure. In some embodiments, the safety monitoring module 310 receives inputs from the data capture module 302, data processing module 304, procedure execution module 306, and/or device control module 308 during execution of the procedure. Module 310 can evaluate such inputs against predefined or dynamic safety constraints. Upon detecting a safety condition—such as excessive force, unexpected motion, loss of sensor integrity, protocol violations, or system faults—the safety monitoring module 310 can be configured generate override signals that restrict, interrupt, or terminate physical interaction, regardless of the current level of autonomy. In some embodiments, safety monitoring module 310 can be configured to transmit an alert notification to a remote device 218 of a human expert that is monitoring the medical procedure. The alert notification is generated in response to detection of a safety condition, system fault, or protocol violation and includes contextual information such as the nature of the detected condition, relevant sensor data, system state information, or a severity indicator. The alert notification enables the human expert to assess the situation and, if appropriate, provide supervisory input, override commands, or other instructions to the computing device 212 and/or other components of system 200. In some embodiments, actions taken by the safety monitoring module 310 cannot be overridden by procedure execution logic generated by module 306 or device control commands generated by module 308.

Turning back to FIG. 2, the computing device 212 can receive data from environment sensors 210 such as images, audio signals, spatial measurements, or environmental condition measurements relating to the patient 204, the robotic device 206, the procedural devices 208, or surrounding workspace. The computing device 212 can also receive data relating to contact force, device pose, energy delivery, physiological measurements, or other procedure-specific parameters from the procedural devices 208. It should be appreciated that data acquired by the computing device 212 from different sensor sources can be time-synchronized, buffered, fused, or otherwise processed to generate a unified representation of procedural state.

The computing device 212 can receive joint-level, actuator-level, or task-level state information from the robotic device 206, and transmit commands, constraints, or interaction objectives to the robotic device 206 for execution of physical interaction with a patient. In some embodiments, the computing device 212 is also coupled to the patient support structure 202 to receive data relating to patient position, orientation, motion, or support configuration, and to coordinate procedural execution relative to the patient support structure. In operation, the computing device 212 can acquire data continuously, periodically, or in response to detected events. The computing device 212 can process such data to support one or more of procedural execution, safety monitoring, authorization condition evaluation, HITL supervision, or autonomy governance. Also, the computing device 212 can generate outputs including candidate actions, control signals, alerts, quality metrics, or authorization decisions, and provide such outputs to the robotic device 206, the procedural devices 208, the environment sensors 210, or other system components.

It should be appreciated that execution of authorized autonomous physical interaction results in a tangible electromechanical transformation of one or more actuators associated with the patient support structure 202, the robotic device 206, and/or the procedural device 208. Such execution causes physical displacement, translation, rotation, articulation, force application, or compliance modulation of the robotic device 206 and/or the procedural device 208 relative to patient anatomy. In certain embodiments, authorization of a candidate action results in generation of actuator control signals that alter motor currents, torque outputs, joint positions, impedance parameters, or other physical control states, thereby producing a real-world mechanical effect at the device-patient interface. The disclosed governance framework therefore directly controls and constrains physical machine operation and does not merely evaluate or present information.

As mentioned above, the system 200 also includes a communications network 214 configured to communicatively couple the computing device 212 to one or more remote computing resources (such as cloud environment 216 and/or remote device 218 operated by a human expert). In some embodiments, the communications network 214 includes one or more local area networks (LANs), wide area networks (WANs), the Internet, cellular or wireless networks, virtual private networks (VPNs), or combinations thereof. The communications network 214 enables exchange of data, control information, and supervisory input between distributed components of the system 200.

The cloud computing environment 216 comprises one or more remote computing nodes and one or more data stores configured to store, without limitation, sensor data, procedural data, protocol definitions, reference databases, trained models, logs, audit records, or historical procedure information. In some embodiments, the cloud computing environment 216 provides scalable storage, computational resources, or shared services that support execution of the medical procedure, post-procedure analysis, model training, or system updates. Data can be transmitted between the computing device 212 and the cloud computing environment 216 continuously, periodically, on demand, or in response to detected events.

As shown in FIG. 2, the system 200 also includes a remote computing device 218 located at a different physical location and operated by a human expert, clinician, or supervisor. The remote computing device 218 can include one or more user interfaces configured to present procedural information, sensor data, images, alerts, or system state information, and to receive supervisory input, authorization commands, or override instructions from the human expert. Such supervisory input can be transmitted to the computing device 212 via the communications network 214 and treated as an authorization condition or control input within the disclosed governance framework.

### AI Algorithm Configuration

An important aspect of the protocol-driven framework for defining and executing an autonomous or semi-autonomous physical interaction as described herein is the integration of artificial intelligence (AI)-based algorithms/models and data processing routines (e.g., AI model(s) 304a of FIG. 3) with the physical system design described in FIG. 2. The integration of AI-based algorithms/models and data processing routines with the system 200 involves two phases: a training phase and an inference phase.

Generally and without limitation, training refers to a computational process by which one or more models, parameters, representations, or decision policies used by the system 200 are derived, adjusted, or refined based on acquired or historical data (also called training data). Training typically involves processing acquired data to identify patterns, relationships, or mappings between inputs and outputs relevant to execution or governance of a medical procedure. In some embodiments, the computing device 212 is configured to train one or more AI-based algorithms/models locally on the computing device. In some embodiments, the computing device 212 can provide training data acquired from one or more devices in the clinical environment to the cloud computing environment 216 for storage, processing, and training of AI models—which can reside in the cloud environment and are accessible to the computing device 212 via a communication interface (such as an application programming interface (API)).

The AI algorithms used for training include, but are not limited to, supervised learning techniques, unsupervised learning techniques, semi-supervised learning techniques, reinforcement learning techniques, imitation learning techniques, or combinations thereof. Training involves learning mappings between sensed inputs and desired outputs, such as device positioning parameters, motion trajectories, interaction forces, procedural state classifications, quality metrics, or recommended next procedural steps. In some embodiments, training includes labeling of data based on protocol-defined endpoints, human expert annotations, or inferred procedural outcomes.

During training, the system typically receives or acquires training data, which include, e.g., data from patient support structure 202, robotic device 206, procedural devices 208, and/or environment sensors 210 during execution of a medical procedure, contextual information, labels, annotations, or outcomes. The system can apply a learning algorithm that adjusts AI model parameters based on the data and evaluate model behavior/output against one or more objectives, such as accuracy, consistency, safety, or adherence to protocol. The system then produces a trained model that can be used later to analyze newly-acquired data and generate predictions, classifications, estimates, or recommended actions. Importantly, AI model training is logically distinct from real-time inference and from execution of physical interaction. Training produces or updates a model, while inference applies the trained model to new data during operation.

Once one or more AI models are trained, the system 200 can proceed to the inference phase, where the trained AI models are applied to analyze acquired sensor data in real time or near real time and, in some cases, to adjust operation of one or more devices in the clinical environment and/or to modify execution of the medical procedure in response to the analyzed sensor data. As mentioned previously, the AI models can analyze sensor data such as, without limitation, data relating to device pose, motion, force, contact state, patient position or movement, environmental conditions, physiological signals, or combinations thereof. Such analysis is used to estimate procedural state, assess data quality, detect deviations from expected conditions, or predict outcomes associated with continued execution. Based on real-time analysis, the AI models can generate inference outputs including candidate actions, control parameters, confidence measures, anomaly detections, or recommendations for modifying execution of the procedure. The computing device 212 can use the AI model outputs to adjust motion commands, interaction constraints, control strategies, or levels of autonomy, or to trigger evaluation of authorization conditions within the governance framework.

In some embodiments, the system 200 can use outputs generated by AI-based analysis to modify operation of the system in response to procedural conditions. Such modifications include adjusting device positioning, motion trajectories, force limits, compliance parameters, sensing configurations, or control modes; transitioning between human-guided, shared-control, semi-autonomous, and autonomous operation; or requesting additional human supervisory input. Modifications can be applied continuously, periodically, or at defined procedural checkpoints.

The system 200 can further use AI-based analysis to identify safety-relevant events, quality degradation, protocol deviations, or unexpected environmental or patient conditions. In response, the system 200 can restrict autonomous physical interaction, escalate to increased human involvement, pause execution, or revoke autonomous operation entirely, in accordance with evaluated authorization conditions.

AI models that can be trained and deployed by the system 200 include, without limitation, classification models, regression models, probabilistic models, sequence models, neural networks, reinforcement learning policies, anomaly detection models, or hybrid models combining learned and rule-based components. In some embodiments, AI models can be combined with rule-based logic, classical control algorithms, or physics-based models to form hybrid systems. The system 200 can train AI models using data acquired from prior procedures, simulations, or human-guided execution, and the system 200 can apply the trained models during execution of a medical procedure to analyze sensor data, generate candidate actions, assess confidence or risk, or support authorization decisions.

In addition, trained AI models can be continuously refined, retrained, and validated over time using output generated during execution of procedures to improve future output and decision-making. In some embodiments, the system 200 can perform model updates offline, between procedures, or during scheduled update cycles. In some cases, the system 200 can incorporate human expert review or approval into the model retraining process. In this manner, the system 200 supports continuous improvement while maintaining separation between model training, real-time inference, and governance of autonomous physical interaction.

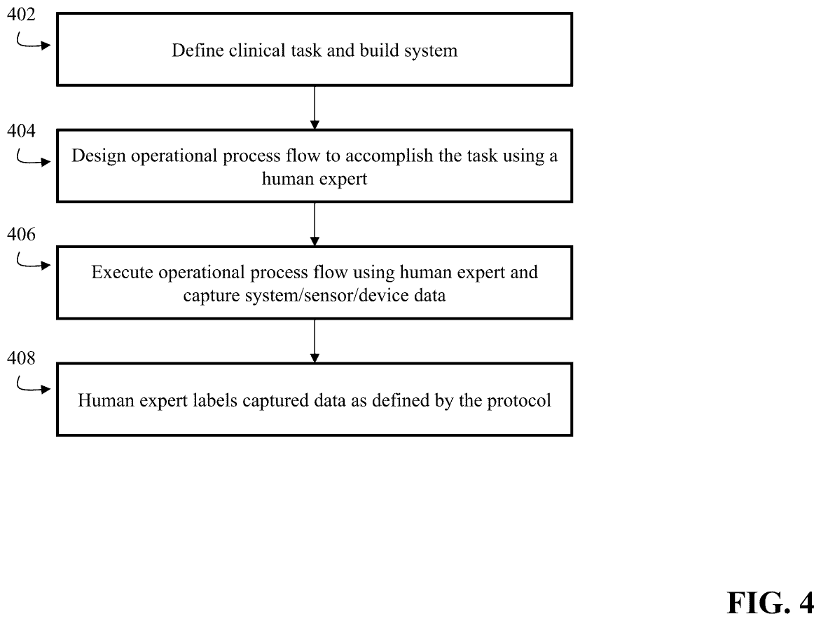

FIG. 4 is a flow diagram of an exemplary method 400 for generating a training dataset to be used in training one or more artificial intelligence (AI) models described herein. At step 402, a clinical task is defined, for example in accordance with a standardized clinical protocol or a clinician-customized procedure, and a corresponding clinical system and environment are established to support execution of the task. The defined clinical task can specify procedural objectives, required data outputs, quality criteria, and success conditions. By way of a non-limiting example, where the clinical task comprises performance of an echocardiographic ultrasound examination, the task definition can reference a professional standard such as an American Society of Echocardiography (ASE) protocol, and a corresponding system configuration (e.g., the system 200 of FIG. 2) is constructed to support execution of the examination.

At step 404, an operational process flow is defined that represents how the clinical task is performed by a human expert. The operational process flow generally specifies a sequence of actions, decisions, device manipulations, and assessments performed during execution of the task, and may reflect best practices, clinical judgment, and procedural heuristics used by skilled practitioners. In the ultrasound example, a sonographer or other trained clinician defines a process flow describing patient preparation, device configuration, probe positioning and manipulation, assessment of image quality, and decisions regarding progression through the protocol.

At step 406, the human expert executes the defined operational process flow while the system concurrently records data associated with execution of the clinical task. The recorded data includes, but is not limited to, system state data, sensor data, device data, and human interaction data. Continuing the ultrasound example, the sonographer performs the procedure by positioning the patient, applying gel/electrodes to the patient, configuring imaging parameters, and manipulating an ultrasound transducer (e.g., manually or using a controlled robotic device) relative to the patient to acquire diagnostic images. During execution, the system may acquire data from procedural sensors (e.g., force or contact sensors associated with the transducer), environment sensors (e.g., images or three-dimensional measurements of patient posture and transducer pose relative to the patient), robotic device sensors, and imaging equipment (e.g., ultrasound images, operational settings, timestamps, and operator inputs or annotations). Data acquisition is typically time-synchronized across sensor modalities and stored for subsequent processing.

At step 408, at least a portion of the recorded data is labeled in accordance with the defined clinical task or protocol to generate training data. Labeling can be performed by a human expert, such as a radiologist or sonographer, and includes associating data samples with procedural states, outcomes, or quality indicators. For example, ultrasound images acquired during the procedure are labeled according to view classification, anatomical structures, measurement targets, segmentation boundaries, or phase of the cardiac cycle (e.g., systole or diastole). In some embodiments, labels further indicate whether acquired data meets predefined diagnostic quality criteria, such as resolution, field of view, depth, gain, or absence of artifacts. In other embodiments, labeling is supplemented or partially automated using computational techniques, protocol rules, or prior trained models.

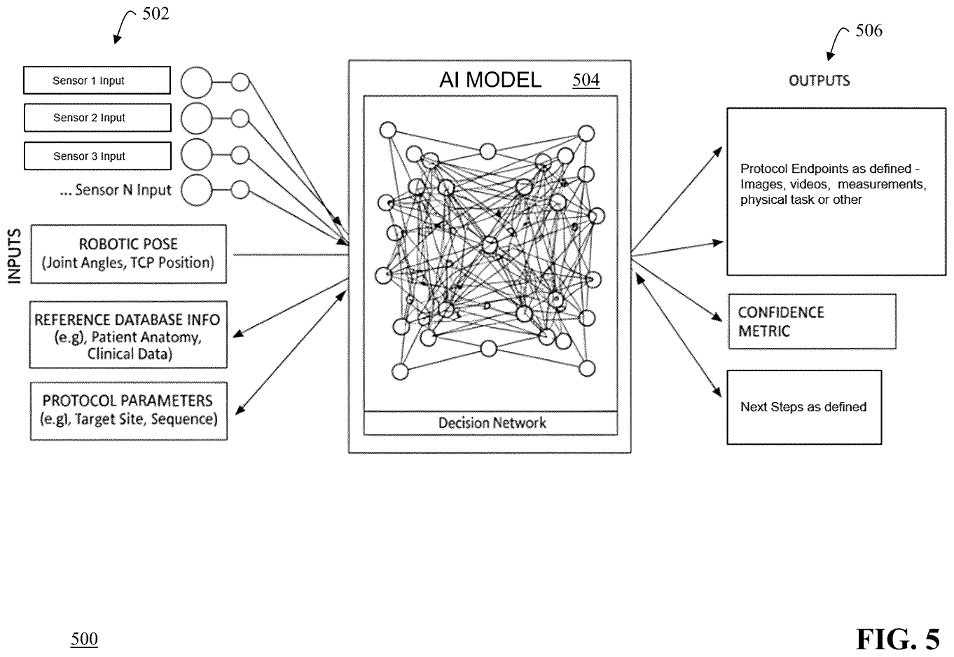

The training dataset generated using the method 400 described with reference to FIG. 4 is used as input for training one or more artificial intelligence (AI) models. FIG. 5 is a flow diagram illustrating an exemplary method 500 for training an AI model 504 using such a training dataset. As illustrated in FIG. 5, training inputs 502 include, but are not limited to, sensor and device data acquired during execution of a clinical task, robotic state or pose information (e.g., joint positions, joint angles, velocities, tool center point (TCP) position or orientation), reference database information (e.g., patient anatomy models, clinical reference data, prior procedure data), and protocol parameters defining procedural objectives, targets, or sequencing constraints.

The training inputs 502 are provided to the AI model 504, which can comprise one or more computational models configured to analyze the inputs and learn relationships between procedural context and desired outputs. As shown in FIG. 5, the AI model 405 includes a neural network; however, other model architectures can be used without departing from the scope of the disclosure. During training, internal parameters of the AI model 504 are adjusted based on the training dataset to enable the model 504 to generate outputs 506 consistent with protocol-defined outcomes, quality criteria, or expert-labeled data.

The trained AI model 504 is configured to produce output data 506 corresponding to one or more aspects of procedural execution or governance. Such output data includes, but is not limited to, inferred protocol endpoints (e.g., images, video frames, measurements, classifications, or completion of defined physical interaction tasks), one or more confidence or quality metrics associated with the inferred outputs, and recommendations or predictions regarding subsequent procedural steps. In some embodiments, the output data 506 is used during execution of a medical procedure to support real-time analysis, candidate action generation, quality assessment, or evaluation of authorization conditions within the disclosed governance framework.

In some embodiments, the AI model 504 generates one or more confidence metrics during training to assess reliability, consistency, or expected suitability of training data, labels, or intermediate model outputs. In the training context, a confidence metric represents a measure of certainty associated with labeled examples, inferred training targets, or model responses to training inputs, and can be used to characterize the quality or representativeness of data used to train the model. Confidence metrics can be derived using statistical measures, model uncertainty estimates, agreement among multiple annotators or models, signal quality assessments, or protocol-defined quality criteria.

During training, confidence metrics can be associated with individual data samples, groups of samples, training batches, or learned representations, and reflect factors such as label reliability, sensor data quality, consistency with protocol-defined expectations, or proximity to known data distributions. In some embodiments, multiple confidence metrics are generated for a given training example, each capturing a different aspect of uncertainty or variability. Confidence metrics can be generated iteratively as training progresses and the metrics can be updated as model parameters are refined.

Confidence metrics generated during training are used to guide training processes and model development. For example, confidence metrics can be used to weight training samples, select or exclude data from training, prioritize additional data collection or labeling, identify outlier or ambiguous examples, evaluate convergence or generalization of the model, or support validation and review of trained models prior to deployment. In this manner, confidence metrics contribute to improving robustness, reliability, and clinical relevance of AI models 504 trained for use within the disclosed system.

As can be appreciated, the trained AI model 504 can subsequently be deployed during execution of a medical procedure to analyze acquired data and to support procedural execution, monitoring, and adaptation. As described above with respect to FIGS. 2 and 3, during the medical procedure, the computing device 212 can capture data from the environment sensors 210, the procedural devices 208, the robotic device 206 and related sensors 206′, the patient support structure 204, or combinations thereof. The computing device 212 can apply one or more AI models (e.g., model 304a) located on the computing device 212 or in the cloud computing environment 216 to the input data to process the data and generate corresponding output.

Based on the received input data, the trained AI model can perform one or more forms of real-time or near real-time analysis. The analysis performed by the trained model can include, but is not limited to, perception tasks (e.g., identifying anatomical features, estimating device pose relative to a patient, assessing contact state), state estimation (e.g., determining a procedural phase or progress relative to a protocol), prediction (e.g., forecasting expected outcomes or interaction forces), or quality assessment (e.g., evaluating data quality or adherence to protocol criteria). As can be appreciated, the specific analytical functions performed by the AI model depend on the clinical task, the type of procedure being performed, and the configuration of the clinical system 200.

As mentioned above, the trained AI model generates output data corresponding to one or more aspects of the medical procedure. The output data can include, but is not limited to, inferred procedural states, estimated parameters (e.g., device positioning or interaction parameters), quality or confidence measures, detected anomalies or deviations, and candidate actions or recommendations for subsequent procedural steps. In some embodiments, the computing device 212 uses the output data from the AI model to adjust control parameters, sensing strategies, or system behavior, or to request additional data acquisition. In other embodiments, the output data is presented to a human operator (e.g., human expert at remote device 218) to support situational awareness or decision-making.

Importantly, the outputs generated by the trained AI model do not themselves authorize physical interaction with a patient. Instead, in some embodiments the outputs can be provided as inputs to a separate safety and governance framework that determines whether, when, and under what conditions autonomous or semi-autonomous physical interaction is permitted. As described in further detail below, such governance incorporates safety constraints, authorization conditions, and HITL oversight to ensure that execution of the medical procedure remains safe, controlled, and clinically appropriate.

### Safety Monitoring and Enforcement

In systems that perform physical interaction with a patient, particularly where automated or semi-automated operation is employed, it is necessary to provide mechanisms that ensure such interaction remains within defined safety boundaries at all times. Accordingly, the disclosed system includes safety monitoring to continuously supervise physical interaction conditions, system state, and operational constraints during execution of a medical procedure. The objective of safety monitoring is to detect conditions under which continued operation presents elevated risk, loss of control, or deviation from intended procedural behavior, and to enable timely restriction, interruption, or modification of system operation to maintain patient safety and system integrity.

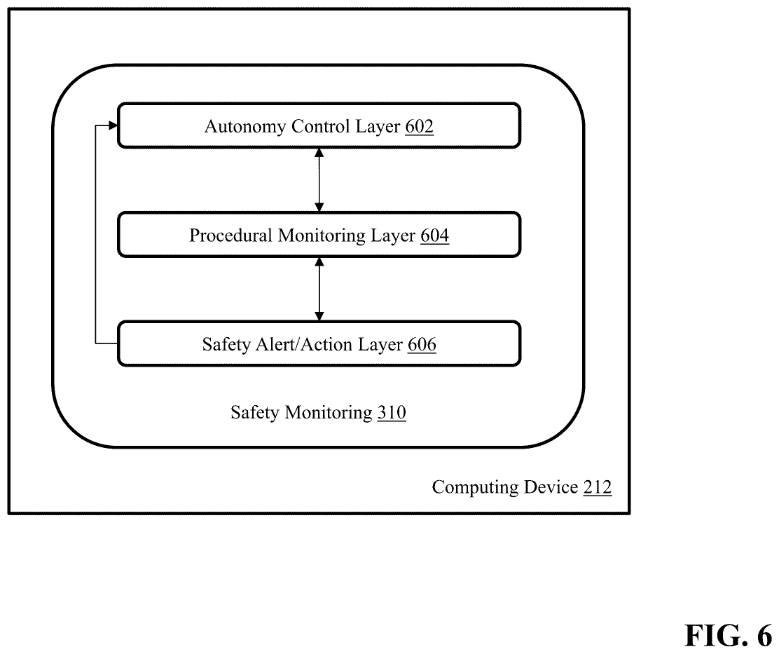

FIG. 6 is a detailed block diagram of the safety monitoring module 310 of FIG. 3. As described above, safety monitoring module 310 can be integrated into the computing device 212 of system 200 for supervising autonomous and semi-autonomous physical interaction between a medical device and a patient. As shown in FIG. 6, safety monitoring module 310 includes an autonomy control layer 602, a procedural monitoring layer 604, and a safety alert/action layer 606.

Generally, the safety monitoring module 310 interfaces with the other software modules 302, 304, 306, 308 of the computing device 212 to receive data from, and send instructions to, the other modules and module 310 operates to detect conditions under which physical interaction should be restricted, modified, interrupted, or terminated in order to maintain patient safety and system integrity. Such conditions include, but are not limited to, deviation from permitted interaction ranges, excessive force or motion, unexpected system behavior, loss or degradation of sensor integrity, communication failures, hardware faults, or violations of protocol-defined safety constraints.

As mentioned above, the safety monitoring module 310 operates continuously and in parallel with procedural execution module 306 and device control module 308. In some embodiments, safety monitoring functions independently of higher-level autonomy execution or decision-making processes and does not rely on outputs generated by trained AI models 304a or other autonomy control algorithms (e.g., as can be implemented in procedure execution module 306 or device control module 308). In this manner, safety monitoring provides an independent layer of protection that remains active regardless of the current level of autonomy or operational mode of the system 200.

As shown in FIG. 6, safety monitoring module 310 includes an autonomy control layer 602 that is configured to adapt operation and control of the system 200 between automated, semi-automated, shared-control, and manually operated modes during execution of a medical procedure. In some embodiments, the autonomy control layer 602 determines, based on procedural context, authorization conditions, and available system inputs, how control authority is allocated among automated control logic, trained AI models, and human operator input. In this manner, the autonomy control layer 602 can enable fully automated execution of certain procedural tasks, cooperative execution in which control is shared between the system and a human operator, or manual operation in which the system provides limited or no automated control. The autonomy control layer 206 can further manage transitions between such modes by modifying control parameters, enabling or disabling automated functions, adjusting levels of assistance, or routing control inputs appropriately, while remaining subject to constraints and overrides imposed by the safety monitoring layer.

Safety monitoring module 310 also includes a procedure monitoring layer 604 that functions as a watchdog during execution of a procedural protocol. In some embodiments, the safety monitoring layer 604 continuously monitors physical interaction parameters (e.g., force, torque, motion, contact state), system state information (e.g., actuator status, sensor health, communication status), and environmental or patient-related safety constraints based upon data received from one or more other modules 302, 304, 306, 308 of computing device 212. Generally, safety constraints can be predefined, protocol-specific, patient-specific, or dynamically updated, and define permitted ranges, thresholds, or conditions for physical interaction.

In addition to enforcing safety overrides based upon defined constraints, the procedure monitoring layer 604 is configured to generate safety-related signals or status indicators that are provided as inputs to the autonomy control layer 602. Such inputs can indicate proximity to prescribed safety boundaries, degradation of operating conditions, or detection of operational characteristics that exceed or approach defined safety limits. Based on such inputs, the autonomy control layer 602 adapts system operation by modifying control parameters, reducing levels of autonomy, transitioning between automated, semi-automated, shared-control, or manual operation, or requesting increased human supervision. In this manner, autonomous operation is permitted only when monitored conditions remain within prescribed safety boundaries, while transitions to restricted or human-guided operation occur proactively in response to detected safety conditions.

The procedural monitoring layer 604 also communicates with safety alert/action layer 606 in the event that safety conditions require human intervention. In some embodiments, upon receiving input from the procedural monitoring layer 604, the safety alert/action layer 606 generates one or more alert notifications for transmission to remote devices and/or initiates safety-related actions. For example, alert notifications can be transmitted by layer 606 to local or remote user interfaces, including devices operated by a human expert (e.g., computing device 218), and such alert notifications can include information identifying the detected condition, associated system state, severity indicators, or actions taken by the system 200. In addition, the safety alert/action layer 606 can trigger adaptation of system processing, including modifying data acquisition rates, adjusting processing priorities, enabling additional monitoring functions, or restricting or suspending certain autonomous processing pipelines of autonomy control layer 602.

In such a configuration, the procedural monitoring layer 604 provides early and continuous assessment of safety-relevant conditions, while the safety alert/action layer 606 coordinates human notification and system-level responses as needed. This separation enables timely escalation of safety concerns, adaptive modification of system behavior, and integration of human supervision, while maintaining the independence and precedence of safety mechanisms over autonomy execution and device control.

### Human-in-the-Loop Autonomy Governance

As illustrated herein, an important facet of the systems and methods is the ability to apply autonomy and governance process flows that regulate how automated and semi-automated functions of the system are enabled, supervised, adapted, or restricted during execution of a medical procedure. Such process flows define how control authority, decision-making responsibility, and operational permissions are dynamically allocated among automated system components and one or more human operators. The autonomy and governance process flows operate in coordination with the procedural protocol, autonomy control, and safety monitoring to ensure that physical interaction with a patient occurs only under appropriate and authorized conditions.

Generally, governance process flows implemented in the system 200 incorporate human-in-the-loop (HITL) oversight as an explicit and integral element of system operation. In this context, a human operator participates in one or more stages of the procedure, including authorization of autonomous operation, supervision of system behavior, validation of system-generated outputs, modification of operating parameters, or intervention in response to alerts or changing conditions. In such embodiments, human involvement can occur locally (i.e., where the human is within the same clinical environment as the patient 204) or remotely (i.e., where the human is viewing the clinical environment from a remote location). Also, such human involvement can be continuous, periodic, or arise based upon specific clinical events, depending on procedural requirements, risk level, or system state.

As mentioned above, during execution of a procedure, the autonomy control layer 602 and the procedure monitoring layer 604 of the safety monitoring module 310 are configured to collect and evaluate governance inputs—including protocol requirements, system state, AI-generated outputs, confidence measures, and safety-related signals—to determine an appropriate level of autonomy that can be afforded to operation of system 200. In some embodiments, the governance process flows implemented in system 200 support multiple operational modes, including fully automated execution, semi-automated or shared-control execution, and manually guided operation. Transitions between such modes can be initiated automatically based on evaluated conditions, manually by a human operator, or through a combination thereof. The governance process flows ensure that such transitions are controlled, traceable, and reversible.

In some embodiments, the governance process flows require explicit human authorization before enabling or expanding autonomous operation of the system 200 for certain procedural steps, regions, or actions. In some embodiments, the governance process flows require reduction of autonomy or escalation to human control when predefined conditions are no longer satisfied, such as degradation of data quality, reduced confidence in system outputs, or proximity to safety boundaries. As such, human input therefore functions as an authorization condition, a supervisory constraint, or an override mechanism within the governance framework.

Furthermore, the autonomy and governance process flows operate subject to safety monitoring and watchdog enforcement. While governance determines whether autonomous operation is permitted and under what conditions, safety mechanisms retain precedence and can independently restrict or interrupt physical interaction regardless of governance state. In this manner, governance process flows regulate when and how autonomy is exercised, while safety mechanisms ensure that autonomy is exercised only when safe to do so.

Through application of these autonomy and governance process flows, the system 200 enables flexible, adaptive, and clinically accountable integration of automated capabilities with human expertise. HITL governance allows the system to leverage automation where appropriate, maintain human oversight where required, and dynamically adapt system behavior throughout execution of a medical procedure.

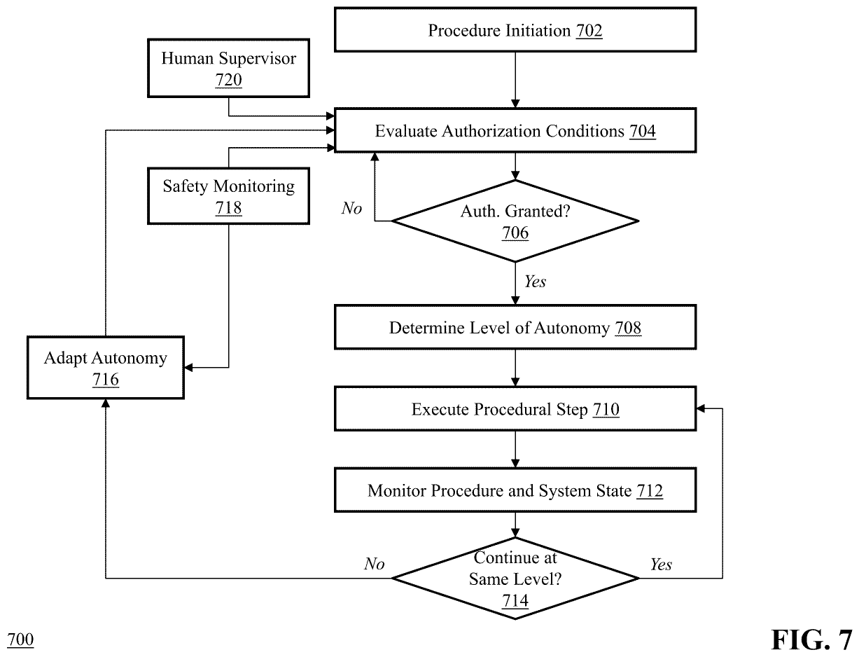

FIG. 7 is a flow diagram of an exemplary method 700 of autonomy and governance with human-in-the-loop and safety monitoring checkpoints using system 200 of FIG. 2. In this method, authorization conditions are evaluated prior to and during execution of a medical procedure to determine permissible levels of autonomous operation, subject to continuous safety monitoring and human supervisory input.

At step 702, the process begins with procedure initiation, in which a procedural protocol, task definition, or clinical objective is selected. For example, the procedure initiation step can include loading a standardized or customized protocol, identifying patient-specific parameters, and initializing system configuration for the procedure. At step 704, the system proceeds to evaluation of authorization conditions. At this stage, the system evaluates whether autonomous or semi-autonomous operation is permitted based on a plurality of inputs. Such inputs include protocol requirements, current system state, outputs generated by trained AI models, confidence or quality metrics, and safety-related signals. In addition, human supervisor input 720 can be received at this stage, for example to authorize, deny, or limit autonomous operation, or to impose additional supervisory constraints.

At decision point 706, the evaluated authorization conditions are assessed to determine whether authorization is granted. If authorization is not granted, the process remains in, or returns to, evaluation of authorization conditions (step 704) until required conditions are satisfied or the procedure is modified or terminated. As can be appreciated, authorization is therefore staged and reversible, rather than a one-time determination.

At step 708, when authorization is granted, the system proceeds to determine level of autonomy. In some embodiments, the autonomy control layer selects an appropriate level of autonomy for the next procedural step (e.g., manual operation, shared or assisted control, semi-autonomous operation, or autonomous operation). As can be appreciated, the selected level of autonomy depends on the nature of the procedural step, current operating conditions, available sensor data, and the extent of human supervisory involvement.

At step 710, the system then performs execution of a procedural step in accordance with the selected level of autonomy. For example, execution involves physical interaction between a robotic device or procedural device and a patient, and includes positioning, motion, sensing, data acquisition, or delivery of a diagnostic or interventional effect.

During and after execution of the procedural step 710, the system at step 712 performs monitoring of procedure and system state. In some embodiments, monitoring includes assessment of physical interaction parameters, device and sensor status, data quality, protocol progress, confidence metrics, and safety-related conditions. As described herein, outputs of this monitoring step can be provided to both the autonomy control logic and to safety monitoring mechanisms of the computing device 212.

At decision point 714, the system determines whether execution should continue at the same level of autonomy. If conditions remain acceptable, the process 700 loop backs to continued execution of subsequent procedural steps under the same autonomy level. If conditions have changed, at step 716 the system can adapt the level of autonomy 716, in which the computing device 212 modifies system operation by reducing or increasing levels of automation, transitioning to shared or manual control, adjusting control parameters, or requesting additional human input.

Throughout the process, safety monitoring 718 (via safety monitoring module 310 of FIG. 3) operates in parallel and independently of the autonomy governance flow. The safety monitoring step 718 can provide inputs to authorization evaluation 704 and autonomy adaptation 716 indicating proximity to, or violation of, prescribed safety boundaries. In addition, safety monitoring can trigger alerts, restrict operation, or interrupt execution regardless of the current autonomy level, thereby ensuring that safety enforcement takes precedence over autonomy decisions.

The process flow 700 of FIG. 7 provides a unified framework in which autonomous operation is continuously governed through explicit authorization conditions, human supervisory input, adaptive autonomy selection, and independent safety monitoring. The process ensures that automation is applied only when appropriate, is dynamically adaptable during execution, and the automation remains subject to human oversight and safety constraints at all times.

### Ultrasound Imaging Illustration

The foregoing sections have described systems, architectures, and process flows for conducting and governing autonomous and semi-autonomous physical interaction in a generalized, device-agnostic, and procedure-independent manner. To further illustrate operation of the disclosed system and to provide concrete context for the interaction of its components, the following section describes an exemplary embodiment in which the system is applied to an ultrasound diagnostic imaging procedure. This embodiment demonstrates how the previously described protocol-driven execution, autonomy control, safety monitoring, and HITL governance mechanisms can be instantiated in a specific clinical setting. As can be appreciated, ultrasound imaging is provided as a representative example, and the described operation does not limit applicability of the disclosed techniques to other physical interactions or medical procedures beyond ultrasound imaging or related applications.

This section summarizes an illustrative embodiment in which the disclosed system is applied to acquisition of diagnostic-quality ultrasound images. In this embodiment, the system operates as a hybrid human-machine system that combines robotic physical interaction, artificial intelligence-based analysis, and human supervisory control to support efficient, repeatable, and safe ultrasound imaging.

In accordance with the previously described architecture, this embodiment employs the semi-autonomous robotic system 200 in which procedural execution is governed by protocol-driven autonomy control, continuous procedure monitoring, and independent safety enforcement. Robotic components (e.g., robotic device 206, procedural devices 208) are configured to perform physically repetitive and precision-controlled probe manipulation tasks, while trained AI models executing on computing device 212 and/or in cloud computing environment 216 assist with perception, state estimation, and procedural guidance. Human expertise is retained through HITL governance exercised by the human expert at remote computing device 218, including authorization of autonomy levels, supervision of execution, and manual override capability.

The embodiment supports multiple ultrasound imaging modes, including A-mode, B-mode, M-mode, and Doppler imaging, and allows dynamic transitions between automated, semi-automated, shared-control, and manually guided operation. In the event that automated or semi-automated execution is restricted or revoked—such as due to safety conditions, reduced confidence, or protocol deviations—the system permits a sonographer to assume direct control of probe positioning and complete the examination.

Safety is enforced throughout the procedure via independent safety monitoring, including limitation of robotic interaction forces to remain within prescribed safety margins that are specific to the targeted anatomy and patient characteristics. Such safety margins can be predefined, protocol-specific, or patient-specific and can be determined using techniques known in the art or described elsewhere in the specification.

The ultrasound embodiment is provided herein to illustrate how the disclosed systems and methods are instantiated in a specific clinical context. As can be appreciated, the described operation does not constrain applicability of the invention only to ultrasound imaging, and the disclosed architecture can be applied to other diagnostic, therapeutic, and/or interventional procedures involving physical interaction with a patient.

The embodiment is described in terms of a preparatory step and a set of scan-time steps, which together provide a standardized, repeatable, auditable, and controllable approach to ultrasound imaging while preserving human-in-the-loop oversight and fallback capability. Throughout the steps described below, physical interaction between a robotic device and a patient is subject to safety monitoring, including limitation of interaction forces to remain within prescribed safety margins. Such safety margins are specific to the targeted anatomy, patient characteristics, or procedural protocol and are predefined, patient-specific, or dynamically evaluated. Determination of particular force thresholds or safety limits can be performed using techniques known in the art or described elsewhere in the specification.

### Step 1: Preparatory Reference Data Generation (Pre-Scan)

Step 1 is a preparatory, pre-scan step in which a reference database is generated to support subsequent scan-time operation. The reference database can include representative procedural flows for ultrasound scanning of one or more organ systems, acquired from multiple subjects and performed under expert human supervision. The reference data is intended to capture variability in anatomy, probe motion, contact conditions, and imaging characteristics, and the reference data is expanded or updated over time as additional data becomes available.

In one illustrative approach, an expert sonographer defines an initial scan region and performs an ultrasound scan of that region using clinically appropriate probe settings, probe motion, and contact conditions. The scan region can be larger than the target anatomy to ensure sufficient contextual coverage across a population. During acquisition, the system records multimodal data including, without limitation, ultrasound image data, visual or depth data of probe pose relative to the patient, motion and pose data of the probe or robotic device, force or torque measurements, and associated timing information.

The acquired data is processed to generate composite representations of the scanned region, such as stitched images, temporally ordered image sequences, or other representations suitable for review and annotation. An expert user annotates anatomical structures, regions of interest, or other clinically relevant features, and such annotations are stored in the reference database in association with the acquired data.

In some embodiments, the preparatory data is further processed to generate joint representations or embeddings that encode relationships among ultrasound data, probe motion, contact conditions, and spatial context. Such representations are generated using one or more machine learning models or other data processing techniques and capture temporal or spatial characteristics of the scanning process. The resulting reference database therefore includes raw data, annotated data, and derived representations, and can be updated as additional data is collected.

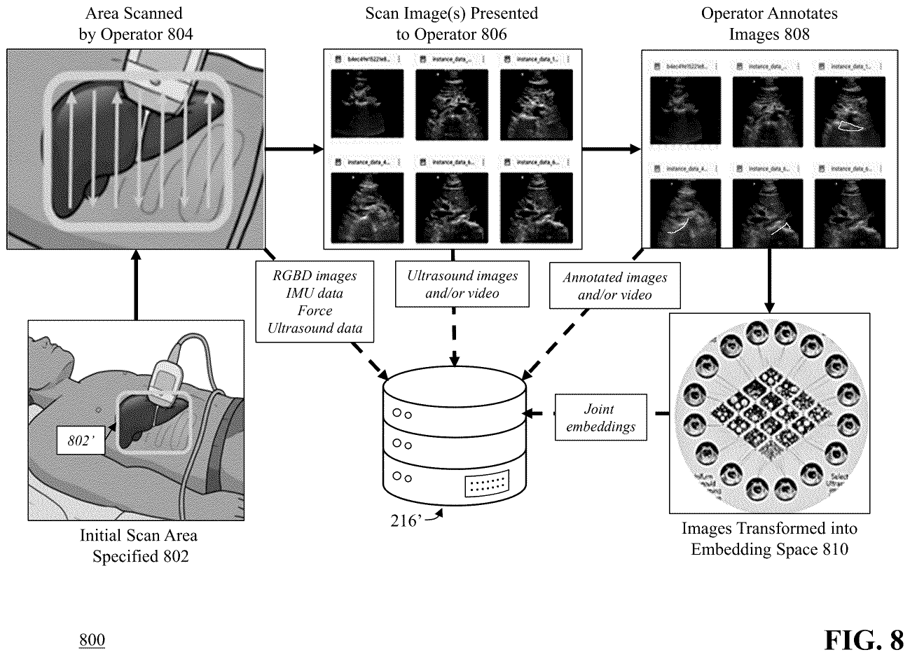

FIG. 8 is an exemplary process 800 for generating a reference database for a selected organ system, performed using system 200 of FIG. 2. As shown in FIG. 8, representative ultrasound scan data is collected by system 200 under expert human control, including acquisition of multimodal sensor data and associated annotations used to construct reference representations stored in a reference data store (e.g., database 216′) for subsequent scan-time operation.

At step 802, an expert human sonographer initiates the reference data acquisition process by selecting an appropriate scan area (box 802′) that encompasses a target organ system (e.g., liver) and, in some embodiments, surrounding anatomical regions. In some embodiments, the scan area 802′ is intentionally larger than the expected anatomical extent of the target organ across a general population in order to account for variability in organ size, position, orientation, and patient morphology. The sonographer configures the ultrasound probe using clinically appropriate imaging parameters for the target organ system, including probe orientation, contact conditions, and device settings.

At step 804, using a controlled scanning technique—such as a serpentine or raster scan pattern—the sonographer operates the robotic device 206 to traverse the scan area with the ultrasound probe 208 that is attached to the robotic device to achieve comprehensive spatial coverage. During scanning, procedural devices 208 coupled to or integrated into the probe record haptic feedback, force sensing, or other physical or motion feedback data to assist the sonographer in maintaining safe and consistent contact between the probe and the patient while positioning and moving the probe.

In parallel, environment sensors 210 such as RGB (red-green-blue), RGBD (red-green-blue plus depth), stereo, lidar, or similar sensors capture spatial context data of the patient and the clinical environment, including recording probe pose, scan depth, and spatial relationship between the robotic device 206, the ultrasound probe 208, and the patient 204. In some embodiments, the environment sensors 210 also capture robotic kinematic data (e.g., from sensors 206′) to capture motion trajectories, orientations, and interaction forces of the robot 206 during the ultrasound scan. In some embodiments, the environment sensors 210 capture image data at a minimum rate of 20 frames per second, providing high-resolution visual context of the external conditions during the scan. For example, an RGBD camera can positioned in the clinical environment so that the entire target area and the movement of the robotic device 206 and ultrasound probe 208 is captured unambiguously.

The computing device 212 also records multimodal data generated during scanning to capture temporal and spatial dynamics of the procedure. In some embodiments, the data recorded by the computing device 212 includes, but is not limited to: visual or depth data from environment sensors 210 (as noted above) that provides external context of the scan region and probe motion; ultrasound image data, captured by an ultrasound machine integrated into or coupled to computing device 212 as frames or sequences corresponding to one or more ultrasound imaging modes (e.g., acquired at no less than five frames per second with a minimum bit depth of 8-bits, these frames provide detailed internal images of the anatomy being examined); motion and interaction data from joints 206′ and/or procedural devices 208, including inertial measurement data, robotic pose information, force, torque, or contact state measurements (e.g., Inertial Measurement Unit (IMU) data, along with pose and force data, recorded at no less than 500 Hz, to capture precise movements of the ultrasound probe, including orientation, angle, and pressure applied during the scan); and timing information recorded by computing device 212 (e.g., each data stream is time-synchronized to enable reconstruction of the scanning process as a coherent temporal sequence—which allows for alignment of ultrasound imagery with probe motion, contact conditions, and spatial context, supporting downstream analysis, joint embedding creation, and representation learning).

At step 806, the computing device 212 and/or cloud computing environment 216 processes the acquired data for review and annotation by the sonographer. In some embodiments, processing includes, but is not limited to, stitching a plurality of ultrasound frames into composite representations of the scanned region, generating ordered frame sequences, or producing other representations suitable for expert evaluation.

At step 808, the computing device 212, cloud computing environment 216, and/or remote computing device 218 presents the images or composite representations to an expert sonographer, radiologist, or clinician. The expert reviews the processed data to identify and annotate anatomical structures within the scan area, including the target organ system and relevant surrounding features. Also, the expert can annotate and record anatomical variations, such as atypical positioning or morphology, while optionally adding descriptive metadata. Such annotations can be used to support later scan-time path planning, localization, or quality assessment. In some embodiments, the expert can annotate and/or explicitly flag observed pathologies or disease indicators. These annotations can be retained for reference or diagnostic purposes but need not be used for scan-time path planning or imaging parameter optimization unless explicitly indicated.

Following annotation, the expert reviewer can assess ultrasound image quality. If the expert deems the image quality insufficient, they can adjust ultrasound settings such as depth, focus, frequency, gain, or acquisition timing, and steps 802, 804, 806 and 808 can be repeated. In some embodiments, steps 802, 804, 806 and 808 of process 800 are repeated and iterated until diagnostic-quality images are obtained, as determined by the expert review.

At step 810, the computing device 212 and/or cloud computing environment 216 generates a joint representation or embedding that encodes a relationship (or relationships) among the collected, time-synchronized multimodal data (e.g., RGBD frames, ultrasound frames, IMU/force/torque data, other optional data such as hyperspectral imaging frames or near-infrared (NIR) sensor data). For example, the joint embedding can represent temporal flow, spatial structure, or similarity relationships across scan sequences. In some embodiments, the computing device 212 and/or cloud computing environment 216 generates the joint embeddings using one or more machine learning models (e.g., AI model(s) 304a) or other data processing techniques.

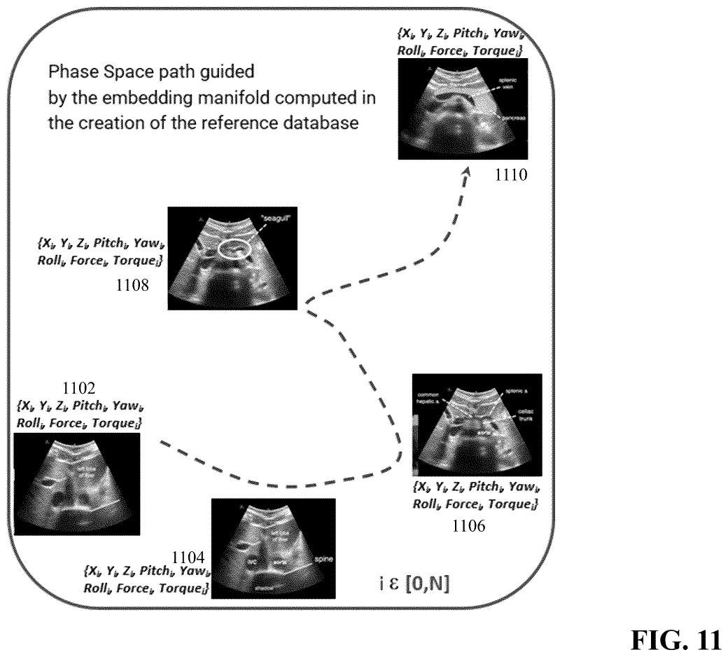

In some embodiments, the embeddings form a manifold or reference space capturing canonical scanning behavior across a representative population for each organ system. FIG. 11 is a diagram of an exemplary workflow process 1100 of planning and updating a scan path based upon the joint embeddings, performed by system 200 of FIG. 2. As shown, representative ultrasound scan states (1102, 1104, 1106, 1108, 1110) acquired during reference data generation are mapped into a multidimensional embedding space that encodes probe pose and interaction characteristics. In some embodiments, each embedded state includes a combination of translational position parameters (x, y, z), rotational orientation parameters (pitch, yaw, roll), and interaction parameters such as applied force and torque, together with associated ultrasound image data. During scan-time operation, the system 200 plans and executes a scan path by traversing the embedding manifold, selecting successive probe states that correspond to representative or optimal scanning configurations learned from the reference database. The illustrated dashed path represents an ordered sequence of probe states i∈[0, N] through the embedding space, where each state corresponds to a distinct ultrasound view and a corresponding set of physical interaction parameters. In some embodiments, transitions between states are determined based on similarity measures, learned transition policies, protocol constraints, or current acquisition conditions.

The same embedding-guided path planning mechanism can be applied across multiple stages of the procedure, including Step 1, where canonical scan trajectories are recorded during reference data acquisition; Step 2, where the embedding is used to guide initial scan-time path planning and adaptive probe motion; and Step 3, where the probe is returned to previously identified target locations for focused imaging or parameter optimization. In some embodiments, traversal of the embedding manifold is updated dynamically based on newly acquired data, quality assessments, safety constraints, or human supervisory input, thereby enabling adaptive, repeatable, and data-driven control of robotic probe motion throughout the procedure.

Turning back to FIG. 8, in some embodiments, the computing device 212 and/or cloud computing environment stores the joint embeddings, together with associated raw data, processed data, and annotations, in a reference data store (e.g., database 216′). As can be appreciated, the reference data store can be updated over time as additional data is collected. Also, the computing device 212 and/or cloud computing environment 216 can access reference data stored in the database 216′ during scan-time operation to support data-driven path planning, similarity matching, and adaptive control for new patients.

### Step 2: Initial Scan-time Acquisition With Human Guidance

Step 2 is an initial scan-time step in which ultrasound data acquisition is performed under HITL governance. In this step, a human operator identifies (or confirms) an initial scan region for the target anatomy using one or more sensor views, such as RGBD images. The robotic device 206 and corresponding procedural devices 208 then perform an initial scan of the region using a predefined or adaptive scan pattern while adjusting probe motion, patient contact conditions, and imaging parameters based on comparison to reference data stored in the database 216′.

In some embodiments, the computing device 212 can perform the comparison between current acquisition images/data and reference images/data using similarity measures, learned models, or other matching techniques. Also, image and system data acquired during Step 2 is presented to the human operator, optionally together with reference examples or quality indicators. The human operator determines whether the acquired data satisfies diagnostic quality criteria or whether re-acquisition or adjustment is required. Based on this input, the system 200 can repeat all or part of the scan, adjust imaging parameters or probe motion, or modify the scan plan. At any point during Step 2, the human operator can assume direct control of probe positioning or system operation as a manual override.

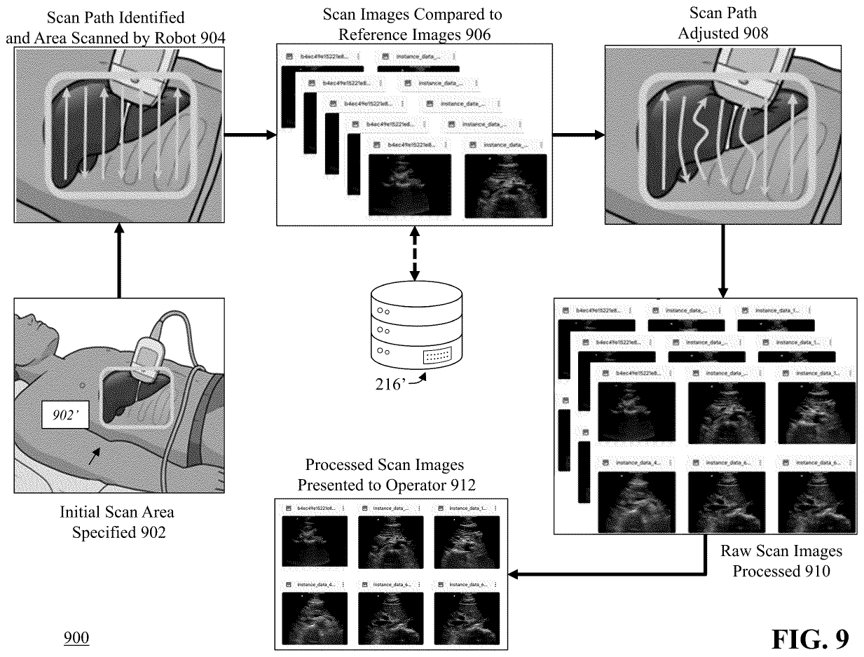

FIG. 9 is an exemplary process 900 for an initial scan-time acquisition, as performed using system 200 of FIG. 2. As shown in FIG. 9, an initial scan region is defined, a robotic system performs an initial scan under HITL governance, and acquired data is evaluated and iteratively refined to satisfy diagnostic quality criteria.

At step 902, a human operator (e.g., a clinician in the same physical environment as the patient or an expert viewing the procedure remotely via computing device 218) identifies and marks an initial scan region 902′ for the target organ system using one or more sensor views, such as RGBD images of the patient captured by environment sensors 210. Generally, the marked scan region defines a spatial boundary within which the robotic device 206 is permitted to operate during initial acquisition and serves as an input to scan planning and autonomy control logic. In some embodiments, the computing device 212 uses images/data from one or more of the environment sensors 210 to map out the initial scan area in high resolution (converting real space coordinates to a coordinate system used by the robotic device 206) and create a proposed scan trajectory and scan path. The computing device 212 can also initialize parameters for the ultrasound probe (e.g., pose, imaging depth, resolution) and create a force map for probe contact during the scan.

At step 904, the robotic device 206 performs an initial scan of the marked region using a predefined or adaptive scan pattern, such as a serpentine or raster pattern. The robotic device 206 can move the probe 208 in a prescribed scan path (e.g., one line of the scan pattern) through the scan area and capture ultrasound images corresponding to the scan path. At step 906, during execution of the scan path and capturing of ultrasound images, the computing device 212 and/or cloud computing environment 216 compares the acquisition images/data with the reference images/data stored in database 216′ to determine whether the acquisition data closely aligns with the reference data. Concurrently, at step 908 the computing device 212 and/or cloud computing environment 216 continuously adapts robotic motion, probe pose, contact conditions, and ultrasound imaging parameters so that location, orientation, force, and movement of the probe—as well as content and attributes of the captured images—closely align with the reference images, parameters, and other data stored in the reference database 216′ (as generated in Step 1 above). In some embodiments, the computing device 212 and/or cloud computing environment 216 aligns the current acquisition data and the reference data using similarity measures, learned models, joint embeddings, or other comparison techniques that integrate multimodal data, including ultrasound imagery, probe motion, and interaction parameters. Such adaptation enables the robotic device 206 to approximate representative scanning behavior while accounting for patient-specific variation. After completing the scan line, the computing device 212 can determine whether to continue with the next scan line in the scan pattern based upon how closely the acquisition data aligns with the reference data. If the alignment is sufficiently close, the computing device 212 and/or cloud computing environment 216 updates the scan trajectory for the next scan line and instructs the robotic device 206 to execute the scan. If the alignment is not sufficiently close, the computing device 212 can adjust the scan parameters for the current scan line and repeat the scan.

At step 910, once the scan pattern is completed and the acquisition data is determined to closely align with the reference data as described above, the computing device 212 and/or cloud computing environment 216 processes the ultrasound images acquired during the initial scan. In some embodiments, processing includes (but is not limited to) stitching ultrasound frames into a composite representation of the scanned region, generating ordered image sequences, or producing other representations suitable for evaluation.

At step 912, the computing device 212 and/or cloud computing environment 216 presents the processed ultrasound images and related scan data to the human operator and/or clinical expert for review. The computing device 212 and/or cloud computing environment 216 can also present one or more reference examples, quality indicators, or confidence measures derived from the reference database 216′ to the human operator. As can be appreciated, by presenting both currently acquired images/data and reference images/data, the system 200 enables direct comparison and supports informed supervisory decision-making.

In some embodiments, the human operator can analyze the initial scan data to determine its diagnostic quality, including but not limited to the following aspects:

Quality of Ultrasound Settings: the human operator assesses whether the ultrasound settings used during the scan (such as depth, gain, and focus) need adjustment to improve image clarity and detail;

Force and Pose Variations: the human operator evaluates variations in the force applied by the robotic device 206 and the pose of the ultrasound probe 208 to ensure optimal contact and angle relative to the scan area; and

Scan Line Path Planning: the human operator reviews the path followed by the ultrasound probe 208 during the scan to confirm that all intended areas have been adequately covered.

If the initial scan data is determined to be insufficient, the human operator can instruct the system 200 to re-acquire ultrasound images and related data for all or part of the scan region. In some embodiments, the human operator can indicate adjustments to one or more system settings or parameters to be used during the re-scan. Adjustments can include, but are not limited to, modifying imaging parameters, probe motion, contact conditions, scan paths, or autonomy settings. As can be appreciated, the initial scanning process can be iterative, with repeated acquisition and review, until the scan data satisfies diagnostic quality criteria or authorization conditions permit progression to a subsequent step.

Also, at any point in the initial scan process, if the robotic device 206 and/or the ultrasound probe 208 fails to meet the required standards, or if there is a specific need for human expertise, the system 200 provides a bypass option that allows the human operator to assume direct control of probe positioning or system operation. For example, manual override can be invoked in response to system limitations, unexpected conditions, safety considerations, or clinical preference, ensuring that robotic operation does not compromise diagnostic quality or patient safety.

Once the human reviewer (and/or the computing device 212 via automated quality assessment) has evaluated and approved the initial scan images and data as satisfying predefined diagnostic quality criteria, the initial scan-time acquisition step is deemed complete. Upon completion, the system 200 can proceed to subsequent steps, such as targeted optimization (Step 3) or output data generation (Step 4), under continued autonomy governance and safety monitoring.

### Step 3: Targeted Optimization of Imaging Parameters

Step 3 is a targeted optimization step performed after completion of an acceptable initial scan (see Step 2 above). In this step, the human operator or another clinician identifies specific locations, features, or regions of interest for further optimization of imaging parameters. In response to indicia from the human operator, the computing device 212 can operate the robotic device 206 to navigate to the identified locations and adjust imaging parameters, probe pose, or contact conditions to improve image quality. It should be appreciated that step 3 is iterative, with repeated acquisition and evaluation against quality criteria, and continues under shared-control or semi-autonomous operation with continued human supervision. As in other steps, the human operator is able to intervene or assume manual control at any time.

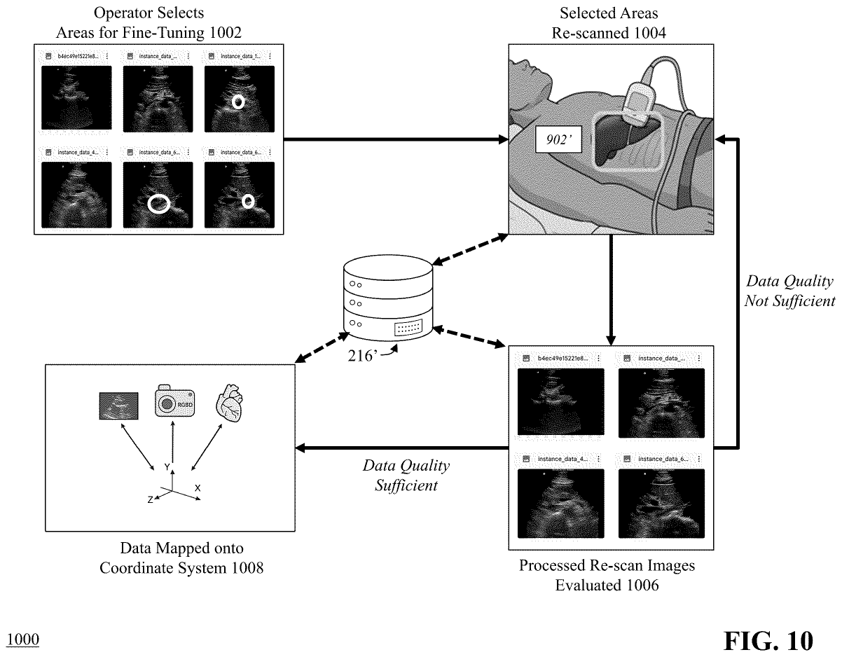

FIG. 10 is an exemplary process 1000 for scan-time procedural flow, performed using system 200 of FIG. 2. As shown in FIG. 10, specific anatomical features, regions of interest, or clinically relevant targets are identified and used to guide focused optimization of ultrasound imaging parameters. This section illustrates how the human sonographer or radiologist collaborates with the system 200 to jointly optimize the ultrasound imaging process.

At step 1002, the system 200 displays initial scan data to a human sonographer, radiologist, or other clinician who reviews the data to identify and annotate one or more target points, regions, or areas requiring further detailed examination. For example, the white circles superimposed on the scan images in 1002 indicate regions of interest (ROIs) that the human expert has marked as areas for follow-up. These targets correspond to anatomical landmarks, regions of diagnostic interest, suspected abnormalities (e.g., lesions, tumors, foreign objects), or other clinically relevant features. In some embodiments, the system 200 designates each anatomical ROI as a patient-specific localized optimization domain. The optimization domain refers to the ROI (or a localized subregion of the ROI) that is designated for optimization of device operating parameters. The optimization domain can be geometrically represented as a bounded region (e.g., rectangular, polygonal, elliptical, or freeform) aligned to anatomical landmarks or internal anatomical orientation inferred during earlier stages. In one embodiment, the patient-specific localized optimization domain may be implemented as a Settings Selection Box (SSB). However, the optimization domain is not limited to a rectangular or explicitly bounded box and may comprise any spatially or functionally defined subregion of acquired data or inferred anatomical space.

The optimization domain functions as a constrained evaluation domain within which acquired data are compared to one or more reference representations. By restricting evaluation to the optimization domain, the system focuses optimization on diagnostically relevant structures rather than global image characteristics. In ultrasound embodiments, the localized optimization domain may correspond to a predicted or inferred acoustic window. An acoustic window refers to a patient-specific spatial region through which acoustic energy can propagate with sufficient quality to visualize a target anatomical structure. The acoustic window may be determined based on external body configuration, rib spacing, thoracic geometry, tissue composition, inferred internal anatomical orientation, or combinations thereof. For example, in an embodiment of a parasternal long-axis (PLAX) cardiac ultrasound procedure, the optimization domain may be aligned with the left ventricle and mitral valve region; in an apical four-chamber embodiment, the optimization domain may encompass ventricular cavities and atrioventricular valves.

At step 1004, upon receiving the annotated targets, the computing device 212 and/or cloud computing environment 216 instructs the robotic device 206 to navigate the ultrasound probe 208 to the optimization domain(s) using positioning and control commands. In some embodiments, controlling the robotic device 206 to navigate the probe 208 to the optimization domain(s) can be performed using spatial context data, robotic kinematics, and/or reference information derived from the reference database.

At each optimization domain, the computing device 212 and/or cloud computing environment 216 performs targeted optimization of imaging parameters and/or probe pose to improve image quality for the identified feature or region. In some embodiments, the imaging parameters can include depth, focus, frequency, gain, beamforming parameters, and acquisition timing. In some embodiments, the imaging parameters are adjusted according to a predefined sequence. In some embodiments, the computing device 212 and/or cloud computing environment 216 optimizes the probe pose according to a 6DOF (six degrees of freedom) pose representation comprising three translational degrees of freedom (x, y, z) and three rotational degrees of freedom (roll, pitch, yaw), defining the spatial position and orientation of the probe relative to a patient reference frame. The targeted optimization can be guided by AI-based analysis, reference data, and/or protocol-specific criteria.

In some embodiments, the optimization domain is dynamically updated as the inferred patient-specific state evolves. Alignment between external sensing data, inferred internal anatomical orientation, and probe pose may be refined iteratively, resulting in corresponding updates to optimization domain location, shape, or extent. The optimization domain may therefore move, rotate, scale, or otherwise adapt in response to updated inference results. The optimization domain is logically coupled to the procedure execution module 306. Image quality metrics, similarity scores, or other evaluation measures computed within the optimization domain are used to determine whether optimized imaging parameters have been identified. The optimization domain thereby serves as a structured interface between anatomical inference and device parameter adaptation. In some embodiments, the optimization domain may be presented to a human operator for confirmation, adjustment, or override. Operator-modified optimization domain boundaries may be incorporated into subsequent inference and adaptive control steps. By establishing a patient-specific coordinate system and landmark-based reference geometry, the system 200 reduces reliance on fixed geometric presets and enables downstream fine-grained optimization within a localized and patient-tailored search region.

In some embodiments, the computing device 212 is further configured to estimate the patient-specific localized optimization domain prior to or during initial probe placement using two-dimensional (2D) and/or three-dimensional (3D) external imaging data acquired from environment sensors 210. The anatomical ROI may be predicted using geometric landmark relationships, learned spatial mappings between surface topology and internal anatomical orientation, regression models, or probabilistic spatial estimators. The predicted anatomical ROI may be represented as a bounding box, surface patch, volumetric region, or parametric search corridor within external anatomical space and mapped into device coordinate space. The anatomical ROI prediction constrains initial probe positioning and limits the operational space for the procedural devices 208 to a sub-region likely to yield diagnostically relevant views.

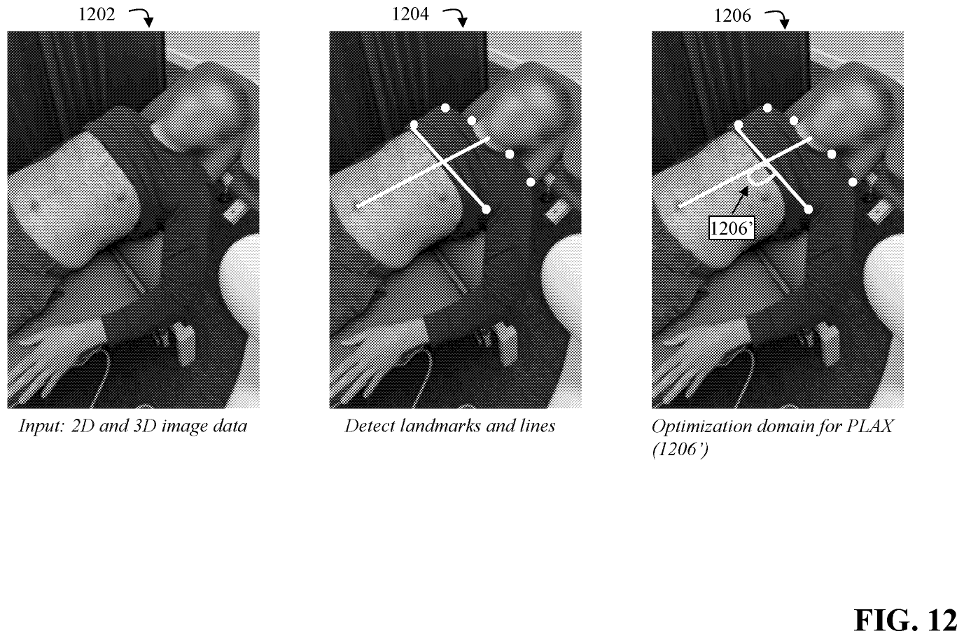

The computing device 212 can perform deterministic geometric estimation of an optimization domain using fused two-dimensional (2D) and three-dimensional (3D) imaging data acquired from environment sensors 210. For example, as illustrated in FIG. 12, the computing device 212 processes external imaging data received from environment sensors 210 (e.g., 2D and 3D image data of patient lying on bed 1202). As shown in annotated image 1204, the computing device 212 uses, e.g., computer vision algorithms to detect anatomical surface landmarks including, but not limited to, shoulders, neck base, axillary (armpit) locations, clavicular contours, and thoracic midline features. In some embodiments, landmark detection may be performed using feature extraction, depth-based segmentation, skeletal pose estimation models, statistical shape models, or combinations thereof.

Using the detected landmarks, the computing device 212 constructs one or more anatomical reference axes, as shown in annotated image 1206. In some embodiments, a longitudinal sternum midline axis is estimated based on detected clavicular alignment and thoracic symmetry, and a transverse axis is constructed by connecting left and right axillary landmarks. These axes define a patient-specific surface coordinate frame aligned with external anatomy.

Based on geometric relationships between the constructed axes and stored anatomical mappings corresponding to a parasternal long-axis (PLAX) acoustic window, the computing device 212 computes a predicted optimization domain 1206′. The predicted optimization domain may be represented as a bounding box, parametric surface patch, angular sector, or volumetric search corridor mapped into device space. The predicted optimization domain constrains initial probe placement and limits the operational adaptation space prior to device-patient interaction. In some embodiments, the optimization domain is presented visually to a human operator, who may confirm, translate, rotate, or resize the optimization domain prior to initiating probe contact. Adjustments made by the operator update the underlying coordinate transforms and corresponding constraints within the operational space for the procedural device 208.

In some embodiments, the computing device 212 continuously or periodically updates the external body configuration inference during execution of the medical procedure. Changes in patient posture, involuntary movement, respiration, or support-surface adjustment can result in updated landmark estimates and corresponding adjustment of the spatial reference frame.

In the illustrative example of FIG. 12, the computing device 212 locates a ROI comprising an optimization domain (e.g., PLAX acoustic window) 1206′ on the patient for the purpose of obtaining an echocardiogram ultrasound. However, other types of localization can be performed for other procedures without departing from the scope of the technology described herein. For example, the inferred external configuration includes estimates of patient orientation relative to, e.g., the patient support structure 202, angular displacement of the torso, elevation or rotation of anatomical segments, and spatial relationship between body landmarks (such as thoracic landmarks useful for localizing an echocardiogram ultrasound) and the robotic device 206 (and/or procedural devices 208). In some embodiments, the computing device 212 accounts for arbitrary patient positioning, including variations in body angle, limb placement, torso rotation, and anatomical proportion differences between patients, when locating the optimization domain for the patient.

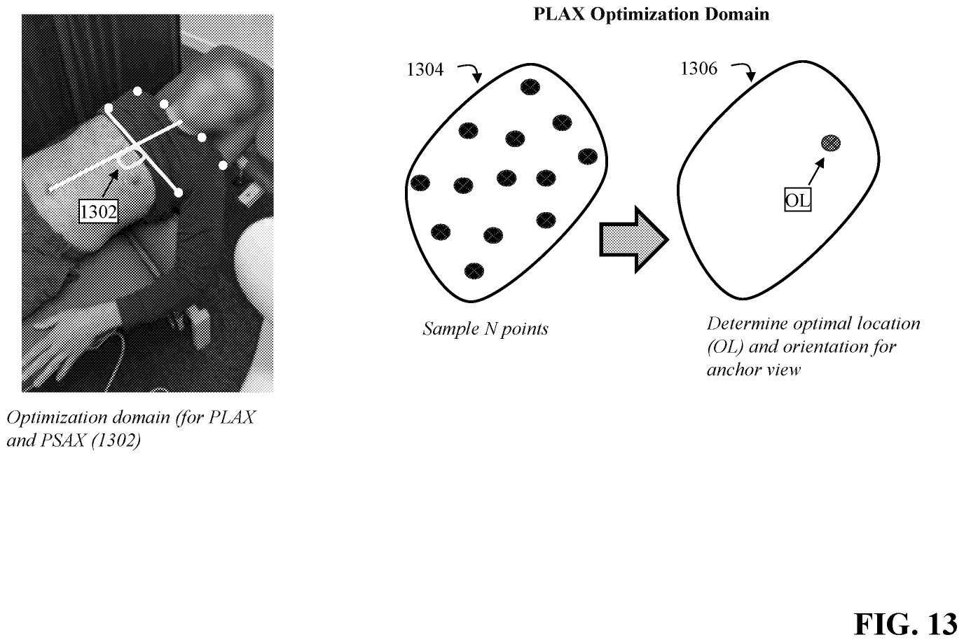

In some embodiments, following estimation of an optimization domain, the computing device 212 performs structured spatial sampling within the predicted optimization domain prior to determination of a final anchor probe pose. For example, as illustrated in FIG. 13, the predicted optimization domain 1302 may be represented as a bounded surface region defined in patient anatomical space and mapped into device space. The robotic device 206 positions the procedural device 208 at a plurality of discrete sampling points (1304) distributed across the window. In some embodiments, the sampling points are arranged according to a grid, quasi-random distribution, stratified sampling pattern, or adaptive exploration sequence within the window boundaries.

At each sampling location, the computing device 212 acquires ultrasound image data and associated interaction parameters, including probe pose, contact force, depth, and orientation. The acquired data is evaluated using one or more similarity or quality metrics relative to stored reference representations corresponding to the desired anchor view. In some embodiments, the computing device 212 uses an algorithm (e.g., a regression-based estimation algorithm) to analyze the collected sampling data for determining an optimal probe location (reference character OL in FIG. 13) and orientation within the optimization domain (1306). The regression model may be deterministic and may estimate a pose vector that maximizes similarity to a reference representation or minimizes alignment error relative to canonical anatomical structures. Such regression can include linear regression, polynomial regression, multivariate regression, surface fitting, or other parameter-estimation techniques.

The estimated optimal probe pose defines an anchor view location within the optimization domain. The robotic device 206 then repositions the procedural device 208 to the estimated optimal pose and reacquires imaging data for confirmation. In some embodiments, the anchor view is presented to a human operator for approval. If the anchor view is not approved, the operator may manually adjust probe pose, and the updated pose may be incorporated into the scanning and/or data analysis. This structured sampling and regression-based optimization approach may be repeated for multiple optimization domains. In the example of a cardiac ultrasound, the optimization domain may include PLAX acoustic windows, parasternal short-axis (PSAX) acoustic windows, apical acoustic windows, subcostal acoustic windows, and suprasternal acoustic windows. As a result the system 200 enables protocol-compliant acquisition of standardized views.

In some embodiments, determination of an optimal probe pose within an optimization domain is preceded by a calibration procedure performed once per system configuration, per probe type, or per procedural protocol. During the calibration phase, the robotic device 206 systematically samples a plurality of probe positions and orientations within a defined optimization domain while acquiring ultrasound imaging data and associated interaction parameters, including contact force, applied pressure, and probe trajectory. For each sampled pose, one or more image quality metrics are computed relative to a canonical reference representation corresponding to a target anchor view (e.g., apical four-chamber or parasternal long-axis).

The calibration procedure characterizes the relationship between probe pose deviations and image quality degradation. In certain embodiments, this relationship is modeled using a regression algorithm that estimates a pose-quality response surface over the optimization domain. The regression model may be linear, polynomial, multivariate, or non-linear, and may incorporate position, orientation, and force components as independent variables. The output of the calibration procedure comprises a fitted regression model that maps probe pose parameters to predicted image quality metrics. For example, in the cardiac ultrasound procedure, the model may compensate for variations in body type, anatomical orientation, optimization domain geometry, and typical heart position within the thoracic cavity.

At scan time, when N sampling points are acquired within the optimization domain for a patient, the calibration-derived regression model is applied to the sampled data to estimate an optimal probe position and orientation that maximizes predicted image quality for the target anchor view. The robotic device 206 then repositions the procedural device 208 accordingly. In some embodiments, calibration may be performed once during system setup, periodically, or upon detection of probe replacement or system configuration change. Calibration data and resulting model parameters may be stored for subsequent retrieval during runtime optimization.

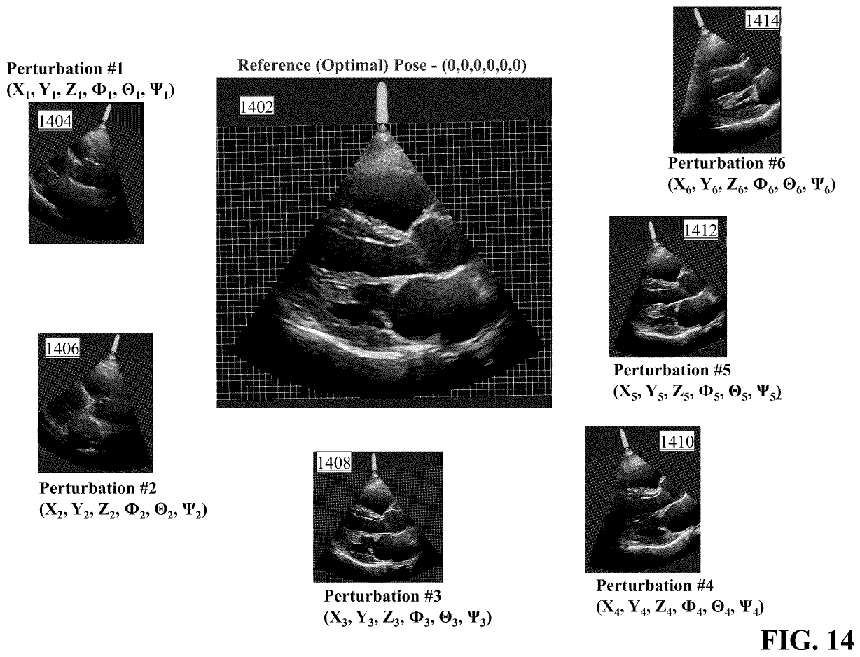

In some embodiments, probe pose optimization within an optimization domain is performed in a six-degree-of-freedom (6-DOF) parameter space comprising three translational components (X, Y, Z) and three rotational components (Φ, Θ, Ψ), corresponding to spatial position and orientation of the procedural device relative to the patient. As illustrated in FIG. 14, a reference optimal pose 1402 may be represented as a pose vector (0,0,0,0,0,0) in a local coordinate frame. Controlled perturbations of the probe pose (e.g., perturbations 1404, 1406, 1408, 1410, 1412, and 1414) may be generated by varying one or more translational or rotational components to produce perturbed poses (Xi, Yi, Zi, Φi, Θi, Ψi). For each perturbation, ultrasound image data and associated interaction parameters are acquired.

Image quality degradation or similarity variation resulting from pose perturbation may be quantified using one or more comparison metrics relative to a reference representation corresponding to a desired anchor view. The system thereby characterizes a local pose-quality response surface in the neighborhood of the reference pose. In some embodiments, a regression model, surface fitting algorithm, gradient estimation procedure, or other optimization method is applied to the perturbation data to estimate a pose update direction and magnitude that improves image quality. The optimization may be performed in full 6-DOF space or in constrained subspaces depending on anatomical context, procedural phase, or safety constraints.

In some embodiments, perturbations are applied sequentially, symmetrically, adaptively, or according to a predefined sampling strategy. Perturbation magnitudes may be bounded by safety limits, anatomical constraints, or device kinematic constraints defined within the operational adaptation space. This perturbation-based modeling enables fine-grained refinement of probe pose, compensating for patient-specific anatomical orientation, thoracic geometry, tissue compliance, and organ positioning variability.

Although FIGS. 12 to 14 illustrate a specific embodiment involving 3D body reconstruction and landmark identification, it should be appreciated that an external configuration inference may be performed using any suitable sensing modality, computational approach, or representational form without departing from the scope of the invention. For example, the first-stage inference need not produce a full body mesh and can instead generate partial, probabilistic, feature-based, or reduced-order representations sufficient to establish a spatial reference for adaptive control of the robotic device 206 and/or the procedural devices 208.

Turning back to FIG. 10, it should be appreciated that imaging at each optimization domain is performed by system 200 in an iterative manner. At step 1006, following acquisition of an image or image sequence, the system 200 evaluates image quality against one or more quality criteria or thresholds, including factors such as resolution, contrast, signal-to-noise ratio, visibility of diagnostic or other target features, or protocol compliance. In some embodiments, the computing device 212 and/or cloud computing environment 216 automatically performs the image quality evaluation based upon configured quality criteria (e.g., minimum thresholds). If the acquired data does not satisfy the quality criteria, the computing device 212 and/or cloud computing environment 216 can determine one or more adjustments to imaging parameters and/or probe pose that are directed to improving image quality. Then, the computing device 212 and/or cloud computing environment 216 can instruct the robotic device 206 to re-acquire the images and related data for subsequent evaluation. In some embodiments, this iterative process continues until the quality criteria are met or until further acquisition is restricted by authorization conditions.

Also, at steps 1004 and 1006, a human operator supervises system operation and image quality. At any time, the human operator is able to intervene, modify parameters, restrict automation, or assume direct control of probe positioning or imaging. This human bypass capability ensures that automated operation does not compromise diagnostic accuracy of the ultrasound examination or patient safety during the procedure.

At step 1008, once all designated optimization domains have images that pass the quality threshold(s), the computing device 212 and/or cloud computing environment 216 records the data generated during targeted optimization—including ultrasound image data, spatial context data, motion and interaction data, and associated annotations in one or more databases (e.g., database 216′). As described herein, the recorded data can include, but is not limited to, RGBD images, ultrasound frames, robotic pose information, inertial measurements, force or torque data, and timing information. The data can be stored for diagnostic review, clinical documentation, training or validation of AI models, or incorporation into the reference database to support future procedures.

### Step 4: Output Data Generation and Registration

Step 4 is an output generation step in which the different modalities of acquired data are registered or mapped into a cohesive data set, i.e., a unified coordinate system or representation. As can be appreciated, the resulting output data set can be used for diagnostic interpretation, reporting, downstream analysis, or archival, among other uses. In some embodiments, the system 200 can incorporate output data generated in this Step 4 into the reference database 216′ to support training, validation, or future procedures.

The system 200 integrates and maps multimodal data collected during the procedure into a common or unified coordinate system. In some embodiments, the multimodal data includes ultrasound image data, spatial context data such as RGB or RGB-D imagery, motion and pose data (e.g., IMU or robotic kinematics), interaction data (e.g., force or torque measurements), and other physiological or environmental sensor data where available. This unified representation reflects probe pose in six degrees of freedom, patient geometry, and spatial relationships among acquired data elements.

As can be appreciated, each data modality can originate in its own local coordinate system based on sensor placement, device geometry, or acquisition characteristics. To generate a unified representation, the system 200 performs spatial and, where applicable, temporal alignment of the data streams. For example, such alignment can include registration of ultrasound image data with external spatial measurements and association of internal anatomical features with external pose and context information. Any suitable registration, transformation, or alignment technique can be used, including but not limited to image-based registration, sensor fusion, learned mappings, or reference-based transformations.

The mappings generated by system 200 associate internal ultrasound imagery with external spatial measurements and anatomical context, thereby enabling consistent spatial registration across modalities and procedural steps. In addition, the unified coordinate representation facilitates downstream analysis, comparison across patients or procedures, data-driven path planning, image quality assessment, and reuse of acquired data during subsequent scan-time operation or training of AI models.

### Other Illustrative Applications

While ultrasound imaging is described herein as a first, non-limiting instantiation of the disclosed systems and methods, the underlying architecture for controlled autonomous and semi-autonomous physical interaction is broadly applicable to a wide range of non-invasive and minimally invasive diagnostic, therapeutic, and interventional medical procedures. The following embodiments are provided as illustrative examples and do not limit the scope of the disclosed subject matter.

Automated or Assisted Cardiopulmonary Resuscitation—in one embodiment, the disclosed systems and methods are applied to automated or semi-automated cardiopulmonary resuscitation. A device configured to physically interact with a patient performs chest compressions or related physical actions in accordance with a resuscitation protocol. Execution of physical interaction is controlled based on evaluated authorization conditions, including protocol compliance, detected patient response, system operational state, and human supervisory input. The level of automation is selectively enabled, limited, shared with a human responder, or suspended in real time, while an independent safety monitoring mechanism retains priority authority to interrupt or modify physical interaction when safety thresholds or fault conditions are detected.

Image-Guided Diagnostic or Interventional Procedures—in another embodiment, the disclosed systems and methods are applied to image-guided diagnostic or interventional procedures, including ultrasound-guided biopsy planning, needle localization, or injection guidance. A diagnostic sensor and associated positioning device are controlled to acquire imaging data and to guide physical interaction relative to clinician-defined regions of interest. Automated positioning or guidance is performed under protocol-defined constraints and subject to authorization conditions that include imaging quality metrics, spatial proximity to anatomical structures, procedural context, and human supervisory approval. Autonomous operation is restricted to defined procedural steps and is suspended or modified when authorization conditions are not satisfied.

Obstetric and Fetal Imaging—in another embodiment, the disclosed systems and methods are applied to obstetric or fetal imaging procedures. A diagnostic sensor is positioned to acquire imaging data according to a standardized or clinician-defined fetal imaging protocol. Automated physical interaction is incrementally enabled for specific scan regions or imaging views and is controlled based on authorization conditions that include image adequacy, patient-and fetus-specific safety constraints, and human supervisory input. Immediate human override capability is maintained throughout the procedure, enabling consistent acquisition while preserving clinical control.

Collectively, these embodiments described herein illustrate that the disclosed systems and methods provide a platform-level framework for controlled autonomous physical interaction that operates independently of specific robotic implementations, sensor modalities, anatomical targets, or procedural workflows, and is applicable across diverse medical environments and use cases.

The systems and methods described herein can be implemented using one or more computing devices configured to execute computer-readable instructions stored in one or more memory devices. Each computing device can include one or more processors, such as general-purpose processors, microprocessors, digital signal processors (DSPs), graphics processing units (GPUs), tensor processing units (TPUs), field-programmable gate arrays (FPGAs), application-specific integrated circuits (ASICs), or combinations thereof.

Memory devices include volatile and non-volatile memory, including random-access memory (RAM), read-only memory (ROM), flash memory, solid-state storage, magnetic storage, optical storage, or combinations thereof. Computer-readable instructions stored in such memory devices implement one or more of the procedural protocol execution logic, authorization evaluation logic, autonomy control logic, safety monitoring logic, sensor data processing, artificial intelligence model execution, and human interface functions described herein.

The computing devices are operatively coupled to one or more sensors, robotic actuators, medical devices, or patient support structures via wired or wireless interfaces. Such interfaces include, without limitation, serial interfaces, parallel interfaces, USB, Ethernet, fieldbus protocols, industrial communication protocols, or medical device communication standards. Sensor data is acquired synchronously or asynchronously and is buffered, timestamped, filtered, or otherwise processed prior to use.

In some embodiments, one or more computing devices are communicatively coupled via a communication network to additional computing resources, including remote computing devices, cloud computing environments, or distributed computing platforms. The communication network includes a local area network (LAN), wide area network (WAN), cellular network, private network, virtual private network (VPN), or the Internet. Network communication supports transmission of sensor data, control signals, procedural parameters, artificial intelligence model data, reference databases, and human supervisory input.

Software components implementing the disclosed functionality are organized as one or more modules, services, processes, threads, containers, or virtual machines. In some embodiments, software components are deployed using containerization, orchestration, or distributed execution frameworks. Software components execute on a single computing device or are distributed across multiple computing devices, including edge devices and cloud-based systems.

Artificial intelligence and machine learning models described herein are trained offline, online, or using a combination thereof, and are executed locally, remotely, or in a hybrid configuration. Model execution includes inference, confidence estimation, quality assessment, and generation of candidate actions. Model outputs are combined with rule-based logic, protocol constraints, and human supervisory input prior to controlling physical interaction.

Human supervisory interfaces include graphical user interfaces, touchscreen interfaces, audio interfaces, haptic interfaces, or combinations thereof, and are provided on local or remote computing devices. Such interfaces support visualization of sensor data, procedural state, alerts, candidate actions, and authorization requests, and receive human input affecting system operation.

The disclosed systems and methods are implemented in software, firmware, hardware, or combinations thereof. Certain functions are implemented using dedicated hardware components, while other functions are implemented using software executed by programmable processors. Allocation of functionality between hardware and software varies by embodiment.

The described computing and implementation environment is exemplary, and variations in hardware architecture, networking configuration, software deployment, and execution environment are employed without departing from the scope of the disclosed subject matter.

As used herein, the terms “comprise,” “include,” and plural forms thereof are open-ended and include the listed elements as well as additional elements not expressly listed. The term “and/or” is open-ended and includes one or more of the listed elements and combinations thereof.

The embodiments described are illustrative and not restrictive. Variations and modifications can be made without departing from the spirit or scope of the disclosure, as will be understood by those skilled in the art.

What is claimed is:

1. A system for performing a medical procedure involving physical interaction with a patient, the system comprising:
a robotic device having a plurality of actuated joints and configured to position a procedural instrument relative to a patient;
one or more sensors configured to acquire, during the medical procedure, sensor data comprising at least one of device pose data, interaction force data, or imaging data associated with physical interaction between the procedural instrument and the patient;
one or more processors operatively coupled to the robotic device and the one or more sensors, the one or more processors being configured to:
execute a procedural protocol defining an ordered set of procedural steps and permitted ranges of physical interaction between the procedural instrument and the patient;
evaluate authorization conditions based on the acquired sensor data and the procedural protocol; and
control actuation of the robotic device based on the evaluated authorization conditions to selectively (a) permit automated movement of the procedural instrument, (b) limit automated movement of the procedural instrument to a subset of degrees of freedom, (c) transition control of the procedural instrument to shared control with a human operator, or (d) suspend automated movement of the procedural instrument; and
a user interface configured to receive supervisory input from a human operator for use in evaluating the authorization conditions or controlling actuation of the robotic device.

2. The system of claim 1, wherein the authorization conditions include one or more of interaction force thresholds, positional constraints, imaging quality criteria, protocol-defined completion states, or combinations thereof.

3. The system of claim 1, wherein the one or more processors are configured to evaluate the authorization conditions continuously during movement of the procedural instrument.

4. The system of claim 1, wherein the robotic device is configured to position the procedural instrument according to a six-degree-of-freedom pose comprising translation along x-, y-, and z-axes and rotation about roll, pitch, and yaw axes.

5. The system of claim 1, wherein the one or more processors are configured to modify the procedural protocol during the medical procedure in response to sensor data or supervisory input from the human operator.

6. The system of claim 1, wherein the one or more processors are configured to generate candidate control actions using a trained machine learning model based on the acquired sensor data.

7. The system of claim 6, wherein the one or more processors are configured to subject the candidate control actions to evaluation of the authorization conditions prior to controlling actuation of the robotic device.

8. The system of claim 1, further comprising a safety monitoring module configured to independently monitor interaction forces and to suspend automated movement of the procedural instrument when a safety threshold is exceeded.

9. The system of claim 8, wherein the safety monitoring module is configured to interrupt automated movement independently of execution of the procedural protocol.

10. The system of claim 1, wherein the one or more processors are configured to map imaging data and device pose data into a unified coordinate system used to guide subsequent positioning of the procedural instrument.

11. The system of claim 1, wherein the procedural instrument comprises an ultrasound probe and the medical procedure comprises a diagnostic ultrasound imaging procedure.

12. A method of conducting a medical procedure involving physical interaction between a medical device and a human body, the method comprising:
controlling a robotic device to position and move a procedural instrument relative to a patient;
acquiring, during the medical procedure, sensor data comprising at least one of device pose data, interaction force data, or imaging data associated with physical interaction between the procedural instrument and the patient;
executing a procedural protocol defining an ordered set of procedural steps and permitted ranges of physical interaction between the procedural instrument and the patient;
evaluating, using one or more processors, authorization conditions based on the acquired sensor data and the procedural protocol; and
controlling actuation of the robotic device based on the evaluated authorization conditions to selectively: (a) permit automated movement of the procedural instrument, (b) limit automated movement to a subset of degrees of freedom, (c) transition control to shared control with a human operator, or (d) suspend automated movement of the procedural instrument.

13. The method of claim 12, wherein the authorization conditions include one or more of interaction force thresholds, positional constraints, imaging quality criteria, protocol-defined completion states, or combinations thereof.

14. The method of claim 12, wherein the one or more processors are configured to evaluate the authorization conditions continuously during movement of the procedural instrument.

15. The method of claim 12, wherein the robotic device is configured to position the procedural instrument according to a six-degree-of-freedom pose comprising translation along x-, y-, and z-axes and rotation about roll, pitch, and yaw axes.

16. The method of claim 12, wherein the one or more processors are configured to modify the procedural protocol during the medical procedure in response to sensor data or supervisory input from the human operator.

17. The method of claim 12, wherein the one or more processors are configured to generate candidate control actions using a trained machine learning model based on the acquired sensor data.

18. The method of claim 17, wherein the one or more processors are configured to subject the candidate control actions to evaluation of the authorization conditions prior to controlling actuation of the robotic device.

19. The method of claim 12, further comprising independently monitoring, using a safety monitoring module, interaction forces and suspending automated movement of the procedural instrument when a safety threshold is exceeded.

20. The method of claim 19, further comprising interrupting, by the safety monitoring module, automated movement independently of execution of the procedural protocol.

21. The method of claim 12, wherein the one or more processors are configured to map imaging data and device pose data into a unified coordinate system used to guide subsequent positioning of the procedural instrument.

22. The method of claim 12, wherein the procedural instrument comprises an ultrasound probe and the medical procedure comprises a diagnostic ultrasound imaging procedure.
